(19) United States
(12) Patent Application Publication
(10) Pub. No.: US 2023/0285353 A1
(43) Pub. Date: Sep. 14, 2023

(54) A CHEMICAL COCKTAIL DRIVING EXPANSION OF MYOGENIC STEM CELLS

(71) Applicant: The Regents of the University of California, Oakland, CA (US)

(72) Inventors: Jun Fang, Los Angeles, CA (US); Junren Sia, Singapore (SG); Song Li, Beverly Hills, CA (US)

(73) Assignee: The Regents of the University of California, Oakland, CA (US)

(21) Appl. No.: 18/005,936

(22) PCT Filed: Jul. 27, 2021
(86) PCT No.: PCT/US21/43292

§ 371 Date: Jan. 18, 2023

Related U.S. Application Data
(60) Provisional application No. 63/058,254, filed on Jul. 29, 2020.

(51) Int. Cl. A61K 31/352; C12N 5/077; A61K 31/444; A61K 31/375

(52) U.S. Cl. CPC A61K 31/352; C12N 5/0658; A61K 31/444; A61K 31/375; C12N 2501/115; C12N 2501/999; C12N 2501/602; C12N 2506/1307

(57) ABSTRACT

We have discovered a chemical cocktail that selectively induces a robust expansion of myogenic stem cells from readily-obtainable dermal cells and from muscle stromal cells. By differential plating and lineage tracing, we show that Pax7+ cells were the major source for chemical-induced myogenic stem cells (CiMCs). We further performed single-cell RNA sequencing (scRNA-seq) analysis to characterize the transcriptomic profile of CiMCs and demonstrate a specific expansion of myogenic cells from heterogeneous dermal cell population. Upon transplantation into the injured muscle, CiMCs were efficiently engrafted and improved functional muscle regeneration in both adult and aged mice. Furthermore, an in situ therapeutic modality using this cocktail was developed by loading the chemical cocktail into injectable nanoparticles, which enabled a sustained release of the cocktail in injured muscle and a local expansion of resident satellite cells for muscle regeneration in adult and aged mice.

### CROSS-REFERENCE TO RELATED APPLICATIONS

This application claims the benefit under 35 U.S.C. Section 119(e) of co-pending and commonly-assigned U.S. Provisional Patent Application Ser. No. 63/058,254, filed on Jul. 29, 2020, and entitled “A CHEMICAL COCKTAIL DRIVING EXPANSION OF MYOGENIC STEM CELLS” which application is incorporated by reference herein.

### TECHNICAL FIELD

The field of the invention relates to compositions and methods for driving the expansion of stem cells.

### BACKGROUND OF THE INVENTION

Skeletal muscle is the most abundant tissue in the human body and serves numerous physiological functions that extend beyond locomotion to other diverse vital functions including signal transduction¹. After injury, skeletal muscles have a capacity for regeneration dependent on resident myogenic stem cells such as satellite cells, which are localized beneath the basal lamina of myofibers and express the paired-box transcription factor Pax7². Quiescent satellite cells are activated upon muscle injury to divide, differentiate, and repair the damaged tissue.³ However, this regeneration capability is compromised by severe acute muscle loss after traffic accidents, blast injuries, combat injuries and surgical resections, or by progressive muscle loss with aging atrophy and inherited genetic diseases such as Duchenne muscular dystrophy (DMD)⁴⁻⁸, resulting in disability and poor quality of life.

Muscle stem cell-based therapies provide promising strategies for improving skeletal muscle regeneration⁹⁻¹¹. However, the muscle regenerative potential is limited by the paucity of autologous muscle stem cells and the need of concomitant immunosuppression for allogenic cells. In addition, muscle stem cell populations that have been expanded in vitro are expensive, time-consuming, and show a markedly diminished efficacy of engraftment¹⁰. Thus, the scarcity of cell sourcing and the lack of an effective method to expand myogenic stem cells are major challenges to using this approach for skeletal muscle regeneration. As an alternative approach, skin dermal cells may provide a convenient cell source to generate skeletal muscle cells via direct cell reprogramming by transfection with the transcription factor MyoD¹², or by dermal stem cell differentiation^{13, 14}. However, dermal cells' myogenic efficiency is relatively poor. Small molecules can modulate cell signaling and thus manipulate cell fate through reprogramming and stem cell differentiation¹⁵. Although several small molecules have been explored to maintain the identity of muscle stem cells¹⁶, enhance myogenic differentiation¹⁷ and promote muscle regeneration^{18, 19}, no small molecule or small molecule cocktail has yet been discovered that can selectively induce and expand myogenic stem cells from dermal cell or muscle stromal cell (MuSC) populations, myogenic stem cells that are useful, for example, in muscle regeneration.

### SUMMARY OF THE INVENTION

As discussed in detail below, it has been discovered that a specific cocktail of chemicals can selectively and efficiently expand myogenic cells from dermal fibroblast-like cells and skeletal MuSCs. The myogenic efficiency of this expansion can be further improved by additional steps such as the selection of primary cells through pre-plating. Importantly, these selectively expanded myogenic cells can be successfully engrafted in vivo to repair pre-injured tibialis anterior (TA) muscles in adult, aged mice as well as in the mdx mouse models of Duchenne muscular dystrophy. In addition, this chemical cocktail can be loaded into injectable nanoparticles, which then enable a sustained release of the cocktail in injured muscle and a local expansion of resident satellite cells for muscle regeneration in adult, aged and mdx mice. Such nanoparticle-delivery of the chemical cocktail in vivo is shown to induce a robust in situ activation and expansion of satellite cells for adult and aged muscle regeneration.

The chemical cocktail (termed the “FR cocktail”) that can efficiently induce and expand myogenic cells from dermal cells and skeletal muscle stem cells in vitro comprises forskolin (F, a cyclic adenosine monophosphate (cAMP) activator) and RepSox (R, a transforming growth factor-β (TGF-β) inhibitor). As described below, methods that use this composition demonstrate the synergistic ability of this combination of molecules to expand myogenic stem cells from dermal cells and skeletal muscle stem cells in vitro. This FR cocktail exhibits a dose-effect to expand myogenic cells from dermal cells and skeletal muscle stem cells in vitro, with the optimal concentration at about 20 μM for both F and R giving the highest yield of myogenic stem cells (63%). In this context, FR cocktail can expand myogenic stem cells from neonatal and adult dermal cells, and from adult and aged MuSCs. Moreover, upon transplantation into the injured muscle, the expanded CiMCs are shown to be efficiently engrafted and improved functional muscle regeneration in a number of in vivo systems.

The invention disclosed herein has a number of embodiments. Embodiments of the invention include compositions of matter comprising amounts of forskolin and amounts of RepSox sufficient to induce and expand myogenic stem cells from dermal cells and skeletal muscle stem cells growing in vitro or in vivo. Certain compositions of the invention are tailored for in vitro use, for example in cell culture systems. Other compositions of the invention are designed for in vivo use, for example in therapeutic methodologies. Illustrative in vitro compositions include media compositions comprising amounts of forskolin and amounts of RepSox sufficient to induce dermal cells and/or skeletal muscle stem cells to become myogenic stem cells in an in vitro culture. Illustrative in vivo compositions include nanoparticles loaded with amounts of forskolin and amounts of RepSox sufficient to induce dermal cells and/or skeletal muscle stem cells to become myogenic stem cells when disposed in an in vivo environment comprising the dermal cells and/or the skeletal muscle stem cells.

Embodiments of the invention also include methods of making and/or using the compositions disclosure herein. Such methods include methods of inducing and/or expanding myogenic stem cells from dermal cells and/or skeletal muscle stem cells in vitro by combining dermal cells and/or skeletal muscle stem cells with amounts of forskolin and amounts of RepSox sufficient to induce and expand myogenic stem cells from the dermal cells and/or the skeletal muscle stem cells. Other methods include introducing amounts of forskolin and amounts of RepSox sufficient to induce dermal cells and/or skeletal muscle stem cells to become myogenic stem cells in vivo by disposing a forskolin and RepSox composition (e.g., disposed in nanoparticles) into the in vivo site (e.g., a site of traumatic muscle injury).

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. A small-molecule cocktail induces myogenic cells from dermal cells. (A) Representative bright-field image of myotubes formed from dermal cells treated with the VCRTF cocktail for 6 days. (B) Troponin T (TnT) staining for dermal cells treated with the VCRTF cocktail for 6 days. (C-D) Quantitative analysis of induced TnT⁺ cells at day 10 after treatment with various combinations of the cocktail. (E) Dose-effect of the FR cocktail. (F) Basic culture medium containing 20 μM F and R with the addition of the listed candidates. (G) TnT staining image of dermal cells induced with an optimal FR medium (basic medium containing 20 μM F and R, 50 μg/ml AA and 50 ng/ml bFGF) for 10 days. *p<0.05, #p<0.0001 (n=3), compared against the conditions in level with the dashed line.

FIG. 2. Characterization of chemical-induced myogenic stem cells (CiMCs). (A) Bright-field images of cells treated with FR medium. DMSO was used as a negative control. (B) Immunofluorescence analysis of skeletal muscle cell markers Pax7, MyoD, MyoG and Myh3 in CiMCs at day 4(D4) and day 8 (D8). (C) The percentage of positive cells in B. (D) qRT-PCR analysis for the indicated skeletal muscle genes of CiMCs at day 8. *p<0.05, **p<0.01 (n=5 for C, n=3 for F).

FIG. 3. Specific upregulation of myogenic gene expression in CiMCs. (A) qRT-PCR analysis for skeletal muscle genes in CiMCs. (B) qRT-PCR analysis for pluripotency genes in CiMCs. (C) Flow cytometry for SSEA1⁺ cells in CiMCs. (D) qRT-PCR analysis for markers of other mesodermal cell types in CiMCs. Myoblasts were included for comparison. *p<0.05, #p<0.0001 (n=3).

FIG. 4. Enriched dermal myogenic cells contribute to chemical-expanded CiMCs. (A) Immunofluorescence staining for Myh3 in RACs, SACs, and HFCs treated with FR medium for 8 days. (B) The percentage of Myh3⁺ cells in A. (C) Immunofluorescence staining for Sox10 and Myh3 in SACs treated with FR medium for 8 days. (D) Immunofluorescence staining for Sox10, Myh3 and FSP1 in HFCs treated with FR medium for 8 days. (E) Immunofluorescence staining for Pax7-FSP1, Pax7-Ki67, MyoD-Ki67 and Myh3 in CiMCs induced from SACs at D8. (F) The percentage of Pax7⁺ cells in CiMCs induced from SACs at day 4(D4) and day 8 (D8) (left) and the percentage of proliferating Pax7⁺ cells based on Ki67 expression (right). (G) The percentage of MyoD⁺ cells in CiMCs induced from SACs at day 4 and day 8 (left) and the percentage of proliferating MyoD⁺ cells based on Ki67 expression (right). (H) Long myotubes that contract spontaneously. The TnT staining of CiMCs cultured in Fb medium for 1 week, revealed a striated pattern in the multinuclear myotubes. Values are mean±SD, **p<0.01 (n=5).

FIG. 5. Dermal Pax7⁺ subpopulation mainly contributes to the expanded CiMCs. (A) Breeding schematic of lineage-tracing Pax7-creER:Rosa26-EYFP mice. (B) Representative images of Pax7 lineage-tracing SACs treated with or without FR medium and 4-OHT for 12 days. BF represents the bright-field image. (C) Selective expansion of SACs from transgenic mice treated with FR medium. (D) Immunofluorescence images of Pax7 and Myh3 in CiMCs from SACs treated with FR medium for 12 days. 4-OHT was added in the Fb medium during cell seeding and 24 hours before the culture medium was replaced with FR medium. (E) FACS sorted day 4 EYFP⁻ and EYFP⁺ cells from transgenic CiMCs treated with FR medium for another 4 days and stained for Pax7 and Ki67. (F) The percentage of Pax7⁺ cells in E. (G) The percentage of proliferating Ki6T cells in Pax7⁺/EYFP⁺ CiMCs at day 0 (D0, 6 hours after plating) and day 4 (D4). **p<0.01 (n=5).

FIG. 6. Chemical-mediated myogenic expansion from adult dermal cells (DC) and MuSCs. (A) Immunostaining images of Pax7 and FSP1 in adult DCs, and adult and aged MuSC s treated with FR medium for 8 days. (B) Percentage of Pax7⁺ cells in A. (C) Immunostaining images of Myh3 in adult dermal cells and adult and aged MuSCs treated with FR medium for 8 days. (D) Percentage of Myh3⁺ cells in C. **p<0.01 (n=3).

FIG. 7. scRNA-seq analysis of chemical-treated dermal cells and endogenous MuSCs. (A) UMAP plot showing the integration of the neo DC and neo DC/FR. Numbers indicate cell percentage in total cells. (B) Pseudotime analysis of myogenic cells from neo DC, neo DC/FR, adult DC/FR and adult MuSC. The color gradient indicates the expression level of the respective genes along pseudotime trajectory. (C) Heatmap showing upregulated genes in the differentiating, quiescent and proliferating myogenic cells. (D) Pseudotime trajectory broken down into the respective samples. Cells from early pseudotime were identified and their un-normalized gene expression data were tested for differential expression testing. (E) Top 20 up and down-regulated gene identified from in Neo DC/FR and endogenous adult MuSC.

FIG. 8. In vivo engraftment of CiMCs promotes muscle regeneration. (A) Maximum isometric tetanic force of CTX-injured TA muscles in adult, aged and mdx mice after transplantation of dermal cells (negative control) or CiMCs at 4 weeks. (B) Representative isometric tetanic force curves of control and CiMC-treated aged TA muscle at 4 weeks. (C) Muscle wet weight of control and CiMC-treated adult, aged and mdx TA muscles at 4 weeks. (D) DsRed-labeled CiMCs and control cells were transplanted into CTX-injured adult, aged and mdx TA muscles for 4 weeks. (E) The number of DsRed myofibers in muscle tissue from D. (F) Average cross-sectional area (CSA) of centrally nucleated myofibers in adult, aged and mdx TA muscles transplanted with either control cells or CiMCs for 4 weeks. (G) Representative myofiber (types I, IIA, and IIB) staining of the adult, aged and mdx TA muscles transplanted with either control cells or CiMCs for 4 weeks. (H) The percentage of distinct myofiber types in G. *p<0.05, **p<0.01 (n=5).

FIG. 9. Drug-loaded nanoparticles promote muscle regeneration. (A) Schematic showing the preparation and injection of drug-loaded particles for in situ myogenic cell expansion and regeneration. (B) SEM image of FR-np and size distribution, as determined by dynamic light scattering. (C) Cumulative release curves of F and R from FR-np obtained by HPLC-mass spectral analysis. (D) The percentage of Myh3⁺ cells in SACs treated with different doses of FR-np. (E) Experimental scheme of CTX injury, nanoparticle (np) injection, and time points samples were harvested for analysis. (F) Representative CMAP curves for vehicle- and FR-np-treated muscles at day 14 (D14) and day 28 (D28). Vehicle refers to np without drugs and served as a control. (G) CMAP amplitude of injured TA muscle treated with FR-np or vehicle alone. (H) Maximum isometric tetanic force of control and FR-np-treated adult and aged TA muscles at 4 weeks. (I) Representative isometric tetanic force curves of control and FR-np-treated adult TA muscles at 4 weeks. (J) Muscle wet weight of control and FR-np-treated adult and aged TA muscles at 4 weeks. (K) Average cross-sectional area (CSA) of centrally nucleated myofibers in TA muscle sections. (L) Representative myofiber staining of control and FR-np-treated adult and aged TA muscles at 4 weeks. (M) The percentage of different myofiber types in L. *p<0.05, **p<0.01 (n=5).

FIG. 10. Drug-loaded particles enhance muscle repair via promoting in situ satellite cell expansion. (A) Immunofluorescence analysis of Pax7⁺ cells in CTX-injured muscle treated with FR-np or vehicle (np without drugs as a control) at day 3 (D3) and day 14 (D14). (B) Quantification of Pax7⁺ cells at different time points after treatment. (C) Immunostaining of Pax7 (green) and Ki67 (red) in CTX-injured muscle treated with FR-np or vehicle at day 3. (D) Lineage-tracing of Pax7⁺ cells in CTX-injured muscle of Pax7-creER-Rosa26-EYFP mice. Image shows numerous EYFP⁺ cells around myofibers in FR-np-treated muscle at day 3. *p<0.05, **p<0.001, n=6.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

As discussed in detail below, the invention disclosed herein has a number of embodiments. Embodiments of the invention include compositions of matter (e.g., culture media compositions, culture media supplement compositions, microparticle compositions and the like) comprising or consisting essentially of amounts of forskolin and amounts of RepSox sufficient to form, induce and/or expand myogenic stem cells from dermal cells and skeletal muscle stem cells growing in vitro or in vivo. Such embodiments include compositions of matter comprising amounts of forskolin and amounts of RepSox sufficient to induce and expand myogenic stem cells from dermal cells and skeletal muscle stem cells growing in vitro. Typically, the composition is in the form of a cell culture media; or a supplement that is added to a cell culture media. For example, embodiments of the invention include a composition of matter comprising a mammalian cell culture media, wherein the cell culture media comprises a supplement disposed therein consisting essentially of forskolin and RepSox. In certain embodiments of the invention, amounts of forskolin and amounts of RepSox are sufficient to create a concentration of forskolin that is between 1 μM and 100 μM (e.g., from 10 μM to 30 μM) and a concentration of RepSox that is between 1 μM and 100 μM (e.g., from 10 μM to 30 μM) in the media. In some embodiments of the invention, the compositions comprise one or more additional agents such as ascorbic acid, basic fibroblast growth factor, and/or a pharmaceutically acceptable carrier. In some embodiments of the invention, the compositions further comprise placental cells, dermal cells and skeletal muscle stem cells; and/or myogenic stem cells.

Embodiments of the invention also include methods of using the compositions disclosure herein. Such methods include methods of growing, inducing and/or expanding myogenic stem cells from placental cells, dermal cells and/or skeletal muscle stem cells and/or other cell sources comprising combining placental cells, dermal cells and/or skeletal muscle stem cells with amounts of forskolin and amounts of RepSox sufficient to induce and expand the population of myogenic stem cells from the placental cells, dermal cells, and/or the skeletal muscle stem cells and other cell sources. In certain embodiments of these methods, amounts of forskolin and amounts of RepSox are sufficient to generate at least 10% more Pax7⁺ myogenic stem cells and/or at least 10% more MyoD⁺ myogenic stem cells growing an in vitro culture (i.e., an at least 10% greater fraction of the cells growing within the cell culture) for at least 4 days as compared to a control (e.g. at least 4 day in vitro culture of dermal cells and/or skeletal muscle stem cells that lacks forskolin and RepSox). Optionally in these methods, the dermal cells and/or skeletal muscle stem cells are further combined with amounts of ascorbic acid and/or basic fibroblast growth factor sufficient to enhance induction and expansion of the myogenic stem cells. In some embodiments of the invention, the methods further comprise disposing the expanded myogenic stem cells at a site of injury in vivo (e.g., a site of skeletal muscle tissue injury).

Another embodiment of the invention is a method of making a mammalian cell culture media, the method comprising combining together typical components used in culturing mammalian cells such as water, fetal bovine sera, a buffering agent, an antibiotic agent and, in addition, a supplement consisting essentially of forskolin and RepSox; such that the mammalian cell culture media in which the cells disclosed herein can grow is made. In typical methods, the cell culture media supplement comprises amounts of forskolin and amounts of RepSox sufficient to induce placental cells, dermal cells or skeletal muscle stem cells growing the cell culture media to form myogenic stem cells. For example, in certain embodiments of the invention, amounts of forskolin and amounts of RepSox are sufficient to create a concentration of forskolin that is between 1 μM and 100 μM (e.g., from 10 μM to 30 μM) and a concentration of RepSox that is between 1 μM and 100 μM (e.g., from 10 μM to 30 μM) in the environment in which the forskolin and RepSox composition is disposed (e.g. a cell culture media, a 1 cm³ region of in vivo tissue proximal to nanoparticles that are releasing forskolin and RepSox or the like). Optionally the methods include adding to the mammalian cell culture media an additional component such as: ascorbic acid; basic fibroblast growth factor; and/or a pharmaceutically acceptable carrier.

Nanoparticle embodiments of the invention can may be made using a variety of diverse biodegradable synthetic and/or natural polymers. Natural polymers include polysaccharides (chitosan, hyaluronic acid, dextran), and proteins (collagen, gelatin, elastin). Biodegradable synthetic polymers include poly(lactic acid) (PLA), poly(glycolic acid) (PGA), polycaprolactone (PCL), polyhydroxyalkanoates (PHA), and their copolymers, poly(ethylene glycol) (PEG) containing polyesters (PLGA-mPEG, PLA-PEG-PLA), polyurethanes (PU), polyamides (polylysine, polyglutamic acid), polyanhydrides, etc. Microparticles or scaffolds also can be made from biodegradable polymers for carrying therapeutic agents. Embodiments of the invention also include compositions of matter comprising nanoparticles (e.g. biodegradable poly (D, L-lactide-co-glycolide) (PLGA)-based blend nanoparticles) loaded with amounts of forskolin and amounts of RepSox sufficient to induce dermal cells and/or skeletal muscle stem cells to become myogenic stem cells when disposed in a localized in vivo environment (e.g. 1 cm³ of tissue) comprising the dermal cells and/or the skeletal muscle stem cells. In certain embodiments of the invention, these nanoparticles further comprise additional agents such as a pharmaceutically acceptable carrier and/or amounts of ascorbic acid and/or basic fibroblast growth factor sufficient to enhance induction and expansion of the myogenic stem cells.

Embodiments of the invention also include methods of introducing amounts of forskolin and amounts of RepSox sufficient to induce dermal cells and/or skeletal muscle stem cells to become myogenic stem cells in vivo, the method comprising disposing a nanoparticle composition comprising forskolin and RepSox into the in vivo site (for example one comprising skeletal muscle tissue); such that dermal cells and/or skeletal muscle stem cells are induced to become myogenic stem cells in vivo.

### Further Aspects and Embodiments of the Invention

In Vitro Induction and Expansion of Myogenic Cells from Dermal Cells by Small Molecules

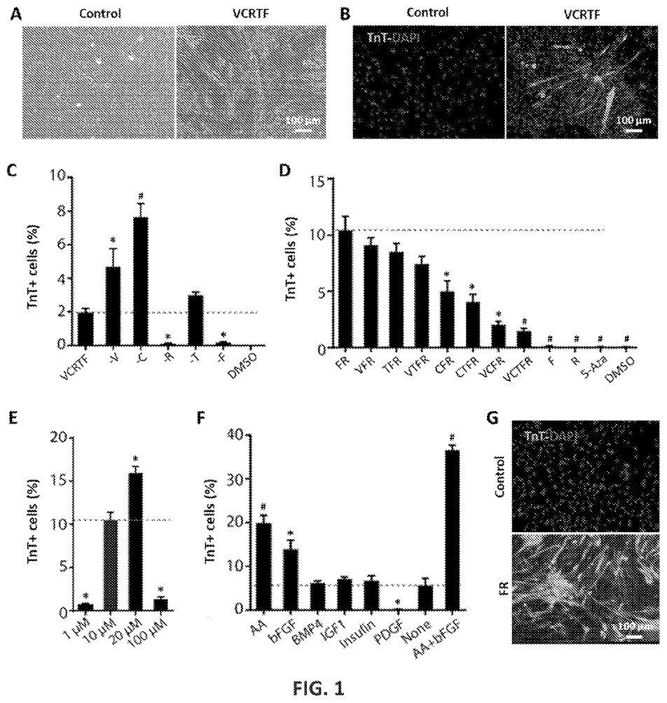

When we used various combinations of chemicals²⁰ to reprogram mouse neonatal dermal fibroblast in culture, we unexpectedly found some myotube-like cells and contracting cell clusters following the treatment of a cocktail of valproic acid, CHIR99021, RepSox, tranylcypromine, and forskolin (VCRTF) (FIG. 1A). Immunostaining showed that these myotube-like cells were positive for skeletal muscle troponin T (TnT) but negative for cardiac myosin heavy chain, confirming the generation of myogenic cells from dermal cells after chemical treatment (FIG. 1B). Before chemical treatment, the dermal cells expressed fibroblast markers FSP1, CD90, PDGFR-α, and neural crest stem cell (NCSC) marker P75. Flow cytometry showed 97.5% of the fibroblast-like cells were PDGFR-α⁺ cells (Fang et al., Nat Biomed Eng. 2021 Mar. 18. doi: 10.1038/s41551-021-00696-y).

To identify the indispensable factors in the cocktail, each compound was omitted, respectively, to generate different combinations of the other four compounds, which were then used to treat dermal cells. Results showed that the number of TnT⁺ cells was significantly reduced when F or R was omitted (FIG. 1C). We next screened different combinations that included F and R. The combination of just F and R (termed “FR cocktail”) maximized the production of TnT⁺ cells, and the addition of other components from the original cocktail reduced the number of TnT⁺ cells or did not improve the efficiency (FIG. 1D).

For comparison, we tested demethylating agent 5-aza-2methylating conditions, which was previously shown to induce transdifferentiation of certain mouse cell lines into skeletal muscle cells²¹. In this study, however, treatment with 5-Aza did not show any significant induction of TnT⁺ cells (FIG. 1D). Dose-optimization studies for FR determined that the combination of 20 μM for both F and R gave the highest yield (˜16%) of TnT⁺ cells (FIG. 1E).

We then tried to further enhance the induction efficiency by adding other factors that have been previously used for culturing muscle stem cells, promoting skeletal muscle cell reprogramming and/or enhancing myogenic differentiation from pluripotent stem cells, including ascorbic acid (AA), bFGF, BMP4, IGF1, insulin, and PDGF^{3, 22·23}. Individually, bFGF (50 ng/ml), as well as AA (50 μg/ml), significantly enhanced the induction of myogenic cells (FIG. 1F). When added together to FR, bFGF and AA synergistically enhanced the induction of TnT⁺ cells to about 37% of the total cell population (FIG. 1F-G). As a result, the optimized medium for inducing myogenesis from dermal cells in vitro contained 20 μM F, 20 μM R, 50 μg/ml AA and 50 ng/ml bFGF, henceforth referred to as “FR medium.”

### Characterization of Chemical-Induced Myogenic Cells (CiMCs)

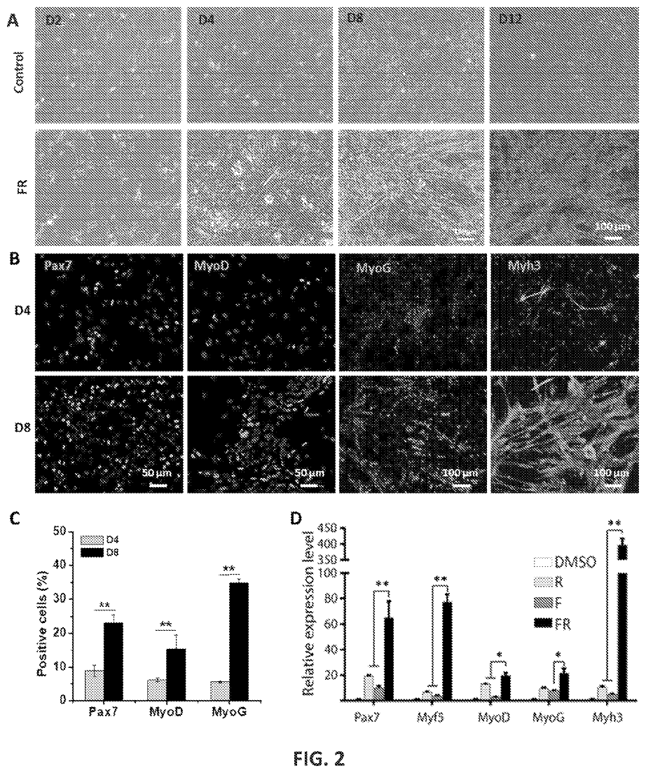

Cell morphology changed gradually following the chemical treatment (FIG. 2A). Notably, dermal fibroblast-like cells treated with FR medium exhibited a slender morphology at day 2, and sparse spontaneously-contracting cells with short myotubes began to appear as early as day 4. The number of myotubes rapidly increased thereafter and gradually became organized into the beating, three-dimensional colonies or clusters. The contracting cell clusters on different days are shown in. No contracting cells or myotubes were detected in control cultures without chemical treatment.

To further characterize CiMCs, the expression of markers known to be associated with different stages of myogenesis was examined by immunofluorescence and quantitative reverse transcription-polymerase chain reaction (qRT-PCR). Satellite cell marker Pax7, muscle progenitor cell marker MyoD, and differentiation markers MyoG and Myh3 were highly expressed in CiMCs (FIG. 2B). The number of Pax7⁺, MyoD⁺, MyoG⁺, and Myh3⁺ cells or myotubes all increased drastically from day 4 to day 8 (FIG. 2C). This observation provides evidence that the chemicals induced and expanded Pax7⁺ satellite cells and/or MyoD⁺ progenitor cells from dermal cells. These cells, in turn, could potentially further differentiate into mature myocytes that fused into multinucleated myotubes. The qRT-PCR analysis confirmed that the induced cells treated with FR showed the highest expression of skeletal muscle genes compared to cells treated with F or R individually (FIG. 2D).

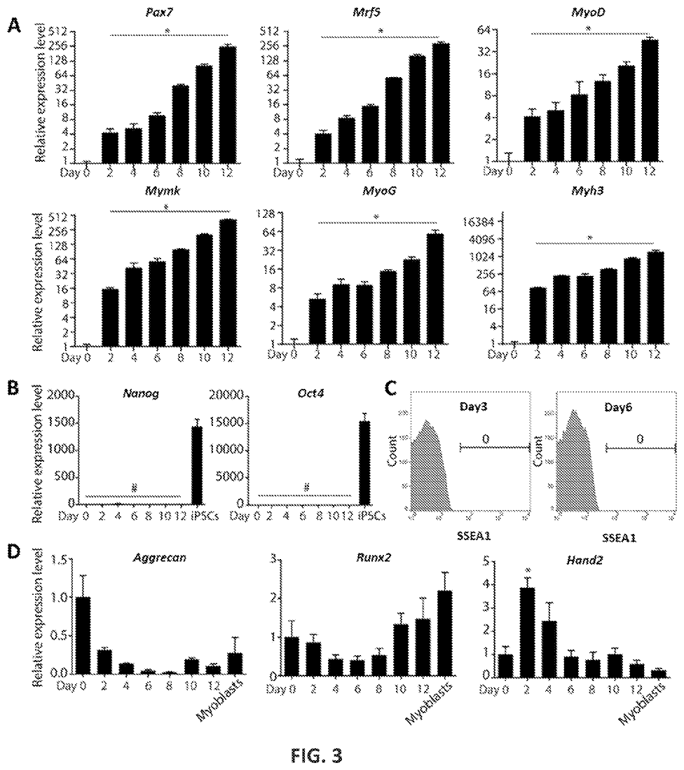

The expression of skeletal muscle genes for CiMCs at different time points was further investigated by qRT-PCR. The data revealed that myogenic genes, including Pax7, Mrf5, MyoD, Mymk, MyoG and Myh3 were all significantly upregulated at day 2 (FIG. 3A). On the other hand, the expression of pluripotency genes Nanog and Oct4 of CiMCs remained undetectable throughout the 12 days of the experiment (FIG. 3B), providing evidence that the cells did not pass through a pluripotent state. Analysis of the cell population on day 3 and day 6 by flow cytometry did not identify any SSEA1⁺ cells (FIG. 3C). Other key markers for mesodermal cell types were also investigated, and markers for cardiomyocytes (Hand2), chondrocytes (Aggrecan) and osteoblasts (Runx2) were not significantly upregulated (FIG. 3D), indicating that only skeletal myogenic cells were specifically induced and expanded in FR medium.

To further investigate the specificity of myogenic induction by the chemical cocktail, the global gene expression of dermal fibroblasts cultured in basal medium versus FR medium for 2 days was analyzed by DNA microarray. The data showed there were 385 up- and 378 down-regulated genes (>two-fold, false discovery rate [FDR]-adjusted p<0.05) for dermal cells cultured in FR medium compared to those cultured in basal medium (Fang et al., Nat Biomed Eng. 2021 Mar. 18. doi: 10.1038/s41551-021-00696-y). Those upregulated by FR medium were significantly enriched for biological processes related to development, while those downregulated were enriched for processes related to cytoskeleton organization and cell adhesion (Fang et al., Nat Biomed Eng. 2021 Mar. 18. doi: 10.1038/s41551-021-00696-y).

CiMCs were Selectively Expanded from Sparse Dermal Myogenic Cells

When the dermal cells at various passages were treated with FR medium, myotubes were clearly generated for the cells in passage 1 and 2, while almost no myotubes formed from the cells of passages 3 or higher (Fang et al., Nat Biomed Eng. 2021 Mar. 18. doi: 10.1038/s41551-021-00696-y). Thus, we postulated that a trace amount of stem cells or precursors might exist in the heterogeneous dermal cell population and contribute to the chemical-induced myogenesis.

We next sought out to determine which primary cell subpopulation could contribute to the chemical-induced myogenesis, by dividing the dermal cells into a rapidly adhering cell (RAC) and a slowly adhering cell population (SAC) using a pre-plating technique. The pre-plating technique selects cells by their differential adhesion to the culture dish surface. Stem cells attach to the culture dish surface weakly and slowly, while the fibroblasts attach more firmly and rapidly. Meanwhile, large hair follicle cell (HFC) clusters were easily separated by low-speed centrifugation during dermal cell isolation. Three cell populations of RAC, SAC, and HFC were examined after overnight seeding by staining with skeletal muscle markers (Pax7, MyoD, Myh3) and skin NCSC or skin-derived stem cell marker Sox10 (Fang et al., Nat Biomed Eng. 2021 Mar. 18. doi: 10.1038/s41551-021-00696-y). Pax7⁺ and MyoD⁺ cells were not or rarely observed in RAC or HFC cultures. In contrast, Pax7⁺, MyoD⁺, and Myh3⁺ cells were detected in SACs, indicating that dermal myogenic cells were enriched in SACs. On the other hand, HFCs showed more Sox10⁺ cells than RACs and SACs.

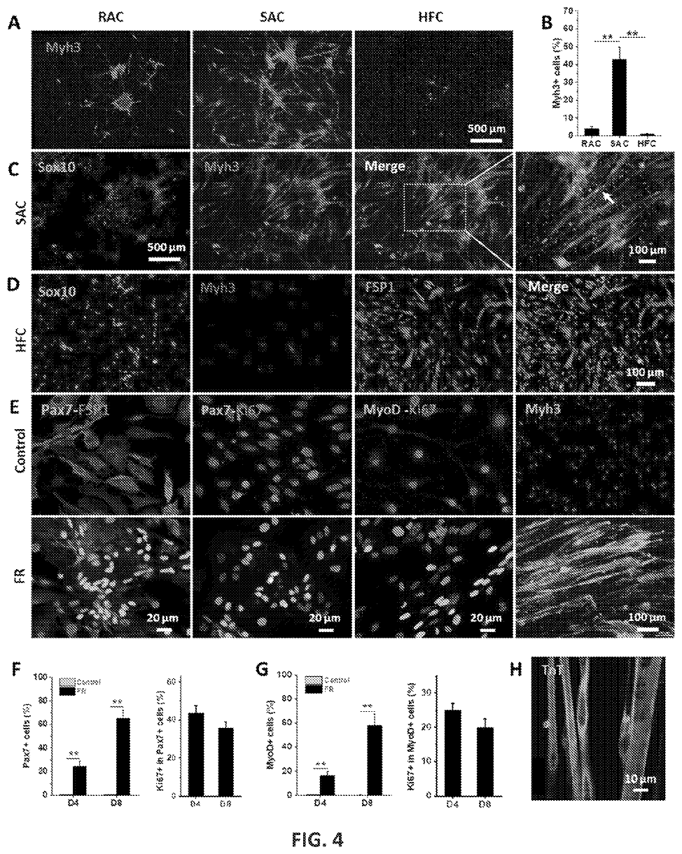

Thereafter, three isolated cell populations were treated with FR medium and characterized to determine their myogenic performances. Strikingly, ˜43% of induced SACs were Myh3⁺, significantly higher than that in induced RACs (4%) and HFCs (0.9%) (FIG. 4A-B), providing evidence that the enriched myogenic cells in SACs potentially enhanced chemical-induced myotube formation. To determine whether sparse NCSCs and HFCs were additional cell sources for chemical-induced myogenesis, we stained for Sox10 in chemical-treated SACs and HFCs (FIG. 4C-D). Results showed that Sox10⁺ cells in SAC-derived cells increased slightly, but very few co-localized with the Myh3⁺ cells. In addition, although more Sox10⁺ cells existed in the HFC-derived cell population, Myh3⁺ cells were sparse. Therefore, these results provide evidence that CiMCs were highly correlated with enriched myogenic cells, and not being selectively expanded from fibroblasts, dermal NCSCs, or HFCs.

Further staining showed that FR treated SACs showed significantly more Pax7⁺ (24.3% at day 4 and 62.5% at day 8) and MyoD⁺ cells (16.2% at day 4 and 57.8% at day 8). In contrast, almost no myogenic stem cells were detected in the controls at day 8 (FIG. 4E-G). At day 4, around 40% of Pax7⁺ cells and 25% of MyoD⁺ cells were Ki67⁺ proliferating cells. In contrast, control preparation contained only proliferating fibroblasts. This confirmed the role of the chemicals in inducing the proliferation of myogenic stem cells. Subsequently, the myogenic stem cells could further differentiate and fuse into multinucleated myotubes with striated pattern (FIG. 4H). In addition, chemical-induced myotubes expressed different types of myosin heavy chains (MHCs), including Myh1E (adult), Myh2 (adult, MHC-IIA), Myh3 (embryonic), Myh 4 (adult, MHC-IIB), Myh7 (adult, MHC-I), Myh8 (neonatal) (Fang et al., Nat Biomed Eng. 2021 Mar. 18. doi: 10.1038/s41551-021-00696-y). Furthermore, CiMCs were passaged every three days to determine whether myogenic potential could be sustained in FR medium. The results showed that myogenic potential persisted in cultures for up to 5 passages and significantly declined afterward, providing evidence that cells could be expanded for 5 passages and potentially used for cell-based therapy (Fang et al., Nat Biomed Eng. 2021 Mar. 18. doi: 10.1038/s41551-021-00696-y)

CiMCs Expanded from Dermal Pax7⁺ Cells

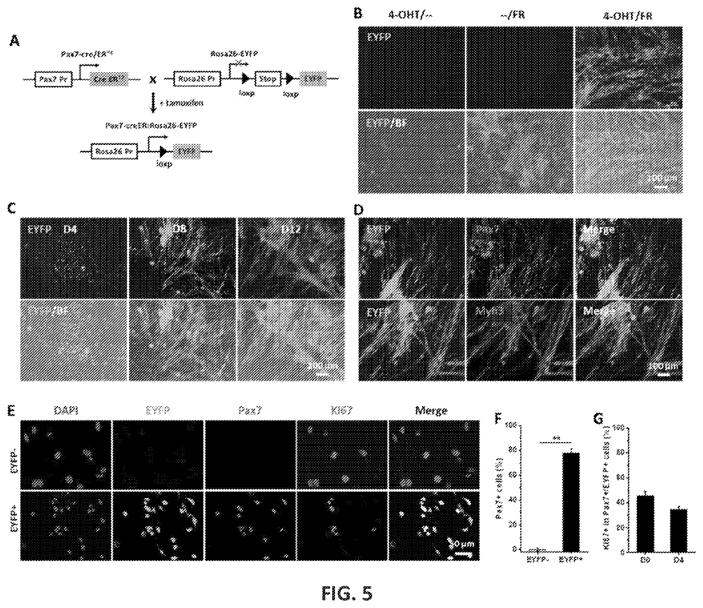

Dermal fibroblast-like cells were isolated from tamoxifen-inducible Pax7-CreER:Rosa26-EYFP transgenic mice to determine the contribution and fate of Pax7⁺ cells in response to the chemical cocktail (FIG. 5A). The EYFP signal was widely detected in CiMCs when the dermal SAC cells were treated with 4-hydroxytamoxifen (4-OHT) and FR cocktail, but no EYFP expression was detected with the treatment of 4-OHT or FR alone (FIG. 5B). The results confirmed the inducibility of EYFP reporter and the reliability of the chemicals to expand myogenic cells. In particular, the EYFP signal was first expressed in individual cells at day 4, and gradually appeared in myotubes/clusters thereafter, confirming that chemicals could expand Pax7⁺ cells, which further differentiated and fused into myotubes (FIG. 5C).

To determine whether the expanded Pax7⁺ cells were derived from existing Pax7⁺ cells or other myogenic precursors, the dermal cells isolated from Pax7 transgenic mice were seeded in Fb medium containing 4-OHT for 1 day to label any existed Pax7-expressing cells with EYFP, and then cultured in Fb medium for 2 more days to remove any residual 4-OHT and prevent any further labeling. The cells were then treated with FR medium for another 8 days. Results showed that the myotubes were EYFP⁺ (Fang et al., Nat Biomed Eng. 2021 Mar. 18. doi: 10.1038/s41551-021-00696-y), indicating that chemical-induced myotubes were mainly derived from originally existed Pax7⁺ cells. Consistently, more EYFP-expressing myotubes formed from induced SACs than from RACs (Fang et al., Nat Biomed Eng. 2021 Mar. 18. doi: 10.1038/s41551-021-00696-y). Further staining demonstrated that almost all expanded myogenic cells and differentiated myocytes/myotubes were EYFP⁺ (FIG. 5D). Additionally, EYFP⁺ and EYFP⁻ cells were sorted by FACS from transgenic CiMCs and further treated with FR medium for 4 days. Immunofluorescence analysis showed that approximately 80% of EYFP cells were Pax7 expressing cells that retained a proliferative capability, as indicated by K167 expression at day 0 and day 4, while no Pax7 expressing cells could be found in chemical-treated EGFP⁻ cells (FIG. 5E-G). Altogether, the results provide evidence that dermal tissue-derived Pax7⁺ cells can be selectively and rapidly expanded via chemical induction.

CiMCs Expanded from Adult/Aged Dermal Cells and MuSCs

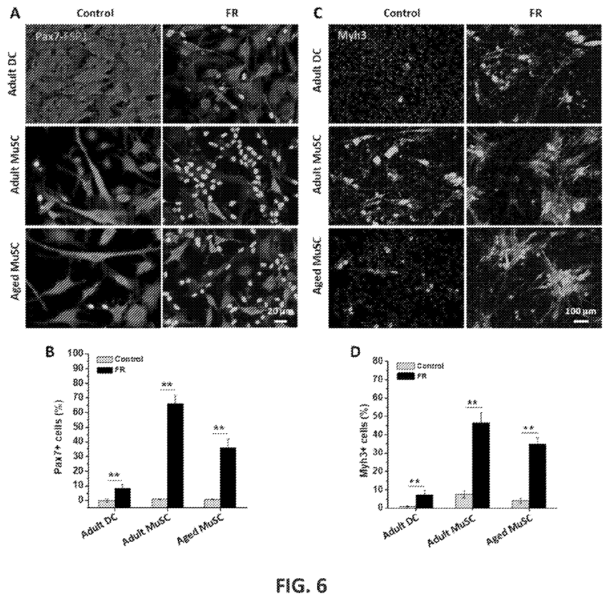

Because of the interest and importance in determining the effect of age on the effective expansion of autologous stem cells for clinical application. We investigated the effects of FR medium on adult dermal cells and on adult and aged MuSCs (FIG. 6). Like our findings with neonatal dermal cells, treating adult dermal cells with FR medium also generated some myogenic cells and myotubes. Adult dermal cells had less Pax7⁺ cells and a lower number of chemical-induced myogenic cells compared to neonatal dermal cells. However, significantly higher numbers of myogenic cells were produced from adult and aged MuSCs (FIG. 6). The chemical treatment induced the proliferation of Pax7 cells from adult MuSCs (31.6% at day 4 and 15.7% at day 8), which yielded approximately 65% for Pax7⁺ cells at day 8 (FIG. 6). Notably, aging reduced chemical-induced myogenic stem expansion to a certain extent, with around 36% of Pax7⁺ cells at day 8. Furthermore, the formation of cell clusters appeared to occur faster in chemical-treated MuSCs compared to untreated MuSCs and chemical-treated dermal cells.

scRNA-seq Analysis

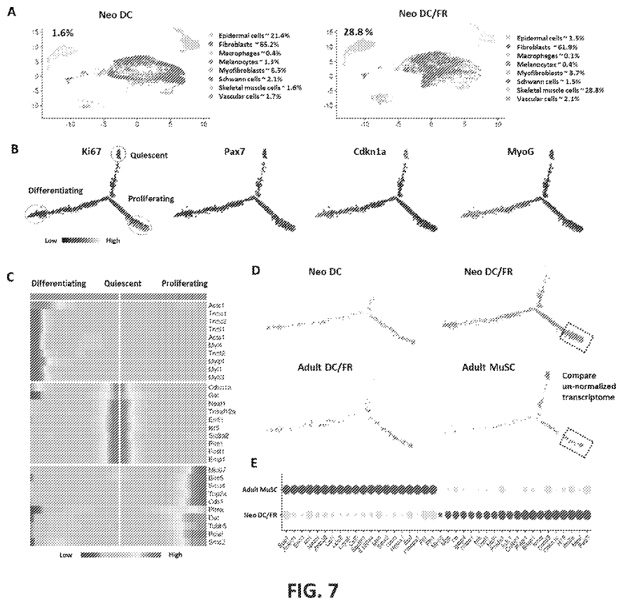

To further characterize CiMCs and the heterogeneity of the dermal cells, we performed scRNA-seq on four samples, including neonatal dermal cells (Neo DC), neonatal dermal cells treated with FR medium for 3 days (Neo DC/FR), adult dermal cells treated with FR medium for 3 days (Adult DC/FR) and endogenous adult MuSC (Adult MuSC). Unsupervised clustering using Seurat²⁴ revealed eight subpopulations in neonatal dermal cells treated with or without chemicals (FIG. 7A). After three days of chemical treatment, the percentage of skeletal muscle cells increased eighteen times, confirming that the chemicals could selectively expand myogenic cells from heterogeneous dermal cells. In addition, both chemical-treated adult dermal cells and endogenous MuSCs were heterogeneous with several subpopulations, while the ratios of the skeletal muscle cells in both samples were much lower than Neo DC/FR (Fang et al., Nat Biomed Eng. 2021 Mar. 18. doi: 10.1038/s41551-021-00696-y). The different cell clusters in all samples were identified by differential marker genes and showed that adult dermal cells had similar gene expression profiles as neonatal dermal cells (Fang et al., Nat Biomed Eng. 2021 Mar. 18. doi: 10.1038/s41551-021-00696-y).

To identify other markers for the Pax7⁺ proliferating cells in the neonatal dermal cells, cells were clustered with a higher resolution to obtain more subpopulations and performed differential expression testing between the Pax7⁺ population and all others. Although three additional genes that were significantly upregulated in the Pax7⁺ population (Spc24, Gnai1 and G0s2), the gene expression distribution plot indicated that Pax7 was the most specific marker for the proliferating myogenic cells (Fang et al., Nat Biomed Eng. 2021 Mar. 18. doi: 10.1038/s41551-021-00696-y).

We then used the skeletal muscle cell cluster data from all four samples, and integrated them computationally and performed pseudotime analysis²⁵. The myogenic cells in neonatal DC, adult DC and MuSC had a similar transcriptomic profile, and co-localized at the various locations of the pseudotime plots, representing myogenic cells at different stages of differentiation and cell cycle. Interestingly, we identified three branches of cells and classified them as quiescent (Pax7⁺/Cdkn1a⁻), proliferating (Pax7⁺/Ki67) and differentiating (Pax7⁻/MyoG⁺) myogenic cells based on marker expression (FIG. 7B-C). In addition, by breaking down the pseudotime trajectory from the original samples, we observed that all four samples (Neo DC, Neo DC/FR, Adult DC/FR and Adult MuSC) had proliferating and differentiating myogenic cells at different stages, while quiescent cells were only found in the adult MuSC (FIG. 7D). Furthermore, we compared the gene expression between proliferating skeletal muscle cells from Neo DC/FR and adult MuSC and found that there were 164 upregulated and 132 downregulated genes in Neo DC/FR; the 20 up and down-regulated genes were shown in FIG. 7G. The gene ontology biological process terms enriched for the upregulated genes in Neo DC/FR were general (Fang et al., Nat Biomed Eng. 2021 Mar. 18. doi: 10.1038/s41551-021-00696-y), indicating that CiMCs exhibited some transcriptomic differences from endogenous muscle cells, but these differences did not point to any specific biological pathways.

### CiMCs Engraftment Improved Muscle Functions in Injured Aged and Mdx Mice

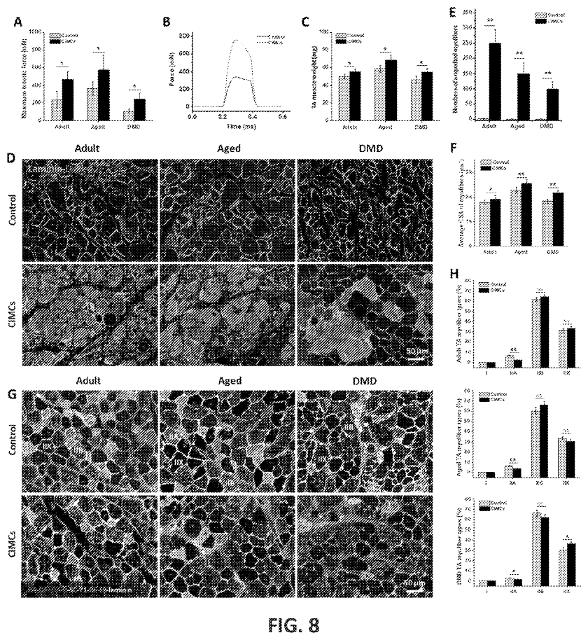

To evaluate the in vivo therapeutic utility of CiMCs for muscle regeneration, CiMCs were collected after 8 days of in vitro expansion in FR medium and injected into the TA muscles that had been pre-injured by CTX in adult, aged and mdx mice. Four weeks post-implantation, force testing was performed to assess the functionality of the regenerated muscle tissue (FIGS. 8A&B). We found that the mean isometric tetanic forces were significantly higher (almost twice) for CiMC-treated TA muscles than for controls in all three mouse models. In general, compared to adult mice, aged mice had larger body weight and muscle mass, and the contraction force was higher, and mdx muscle had the lowest contraction force. Consistently, all muscles were excised following force testing and weighed, and showed that the wet muscle weights were significantly higher for CiMC-treated TA muscles than for controls in all three models (FIG. 8C).

To determine the efficiency of CiMCs engraftment, DsRed-labeled CiMCs were transplanted in injured muscles. Four weeks after cell transplantation into all three animal models (adult, aged and mdx mice), CiMCs were integrated and formed newly regenerated myofibers of various fiber sizes with central nuclei (FIG. 8D-E). Conversely, the contralateral TA muscles transplanted with DsRed-labeled dermal cells did not have DsRed-positive myofibers. Remarkably, numerous dystrophin-positive myofibers were detected in the CiMCs-grafted mdx muscles, while no dystrophin-positive fibers were found in the controls (Fang et al., Nat Biomed Eng. 2021 Mar. 18. doi: 10.1038/s41551-021-00696-y). These results demonstrated that the transplanted CiMCs maintained their myogenic capability and engrafted into regenerated muscles, particularly promoting regeneration of aged and DMD muscles.

On the other hand, the histological analysis of adult, aged and mdx muscles revealed that the average cross-sectional area (CSA) of myofibers was significantly increased for CiMC-treated TA muscles than corresponding controls, especially for aged and DMD muscles (FIG. 8F). Additionally, fibrosis was evidently more severe in both aged and mdx muscles for the control groups, compared to that of injured adult muscles, while CiMC-treated skeletal muscles exhibited significantly lower muscle fibrosis than the controls in all three models (Fang et al., Nat Biomed Eng. 2021 Mar. 18. doi: 10.1038/s41551-021-00696-y). Furthermore, CiMCs-treated muscles exhibited significantly fewer macrophages than the controls in all three models (Fang et al., Nat Biomed Eng. 2021 Mar. 18. doi: 10.1038/s41551-021-00696-y). Taken together, these results provide evidence of the ameliorative effect of CiMCs on fibrosis and inflammatory response also contributed to muscle regeneration.

Skeletal muscle fibers are broadly classified into four myofiber types, including slow-twitch type I, and fast-twitch type IIA, IIB and IIX, and the fiber composition play a critical role in determining muscle function²⁶. Thus, we analyzed the myofiber type composition in regenerated adult, aged and DMD TA muscles 4 weeks after cell injection. Remarkably, three types of fibers (type IA, type IIB and type IIX fibers (unstained by any marker, by exclusion)) were present in all the TA muscles, with the exception of type I fibers. Type IIB myofibers were the most abundant myofiber type in all regenerated TA muscles but had no significant difference between CiMC-treated and control groups. However, type IIA myofibers were significantly fewer in all CiMC-treated TA muscles compared to control groups (FIG. 8G-H), providing evidence that better regenerated TA muscles have less Type IIA fibers.

### Drug-Loaded Nanoparticles for In Situ Muscle Regeneration

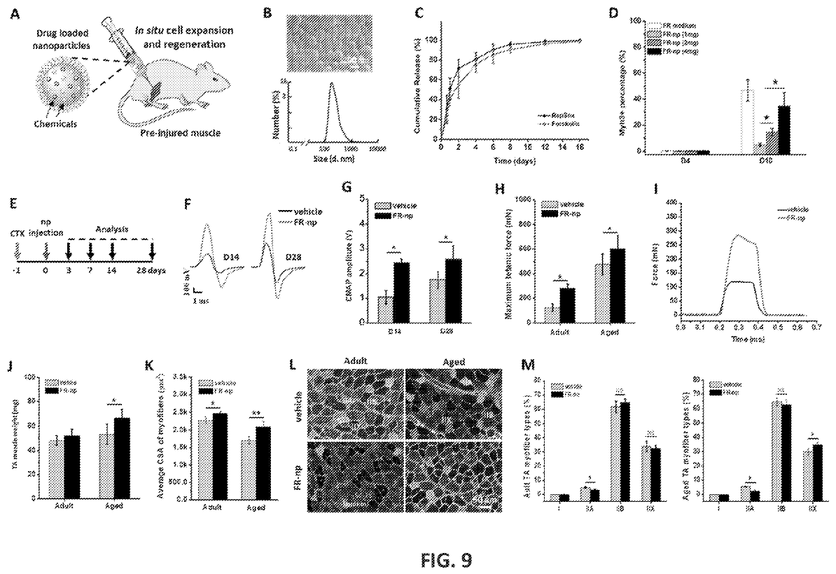

We then explore an in-situ approach to delivering the chemical cocktail to induce myogenic cells in the injured muscle. A delivery platform to control the local release of the chemical cocktail was developed to achieve in situ expansion of resident myogenic cells in injured muscles (FIG. 9A). Both drugs (F and R) were loaded into biodegradable poly (D, L-lactide-co-glycolide) (PLGA)-based blend nanoparticles, hereafter referred to as “FR-np”. Scanning electron microscopy (SEM) showed that the FR-nps were uniform round spheres with an average diameter of 427 nm and polydispersity index (PDI) of 0.24 (FIG. 9B). Both chemicals were gradually released over a two-week time period, as determined by high-performance liquid chromatography-mass spectrometry (HPLC-MS) (FIG. 9C). To further verify whether the drug-releasing particles could selectively expand the myogenic stem cells for myogenesis, we treated SACs with different doses of FR-nps and found a significant increase in Myh3 cells at day 10, with higher doses of FR-nps producing more myocytes or myotubes, thereby recapitulating the myogenic-inducing effects of the released chemicals (FIG. 9D).

To verify the in vivo effect of drug-loaded nanoparticles on muscle regeneration, TA muscles of adult and aged mice were CTX-injured as previously described but were subsequently injected with FR-nps rather than cells (FIG. 9E). At the beginning, to visualize the distribution of injected nanoparticles in injured muscles, green fluorescence-labeled nanoparticles were injected and showed that the nanoparticles were locally and evenly distributed within the TA muscle two days after injection (Fang et al., Nat Biomed Eng. 2021 Mar. 18. doi: 10.1038/s41551-021-00696-y), but gradually degraded and became non-detectable after a month. We injected 1 mg FR-np for each TA muscle repair, based on several considerations including the amount of FR-np to achieve high myogenic induction in vitro condition, an estimated TA muscle volume, a long-term release period (3-4 weeks) and the well-tolerated amount of nanoparticles in the muscle tissue²⁷. After the injection of drug-loaded nanoparticles, muscle functions were monitored and showed that FR-np-treated adult TA muscles had significantly higher sciatic compound muscle action potential (CMAP) amplitudes than the vehicle treatment (i.e., np alone as a control) at week 2 and week 4 (FIGS. 9F&G). Similar to the beneficial effects of CiMCs transplantation, the mean isometric tetanic forces at week 4 were also significantly higher for the FR-np-treated TA muscles than controls in both adult and aged mice (FIGS. 9H&I), as were muscle weights (FIG. 9J) and average myofiber CSA (FIG. 9K), with a significantly lower proportion of Type IIA fibers present than the controls (FIGS. 9L&M). Thus, these results demonstrated that the drug released nanoparticles enhanced in situ TA muscle regeneration and repair.

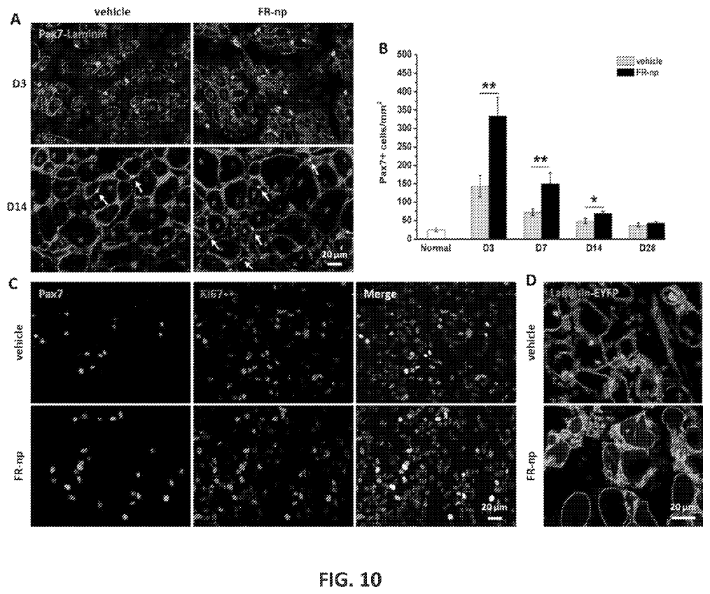

We next determined whether drug-loaded nanoparticles specifically expanded Pax7⁺ satellite cells in situ and accelerated muscle regeneration as FR did in vitro. Immunofluorescence staining showed significantly more peripherally localized Pax7⁺ cells around degenerated or regenerating myofibers in the FR-np-treated muscles compared to control (FIG. 10A). By quantifying the number of Pax7⁺ cells in the region of regenerating myofibers (FIG. 10B), we found that in both FR-np- and vehicle-treated muscle, Pax7⁺ satellite cells rapidly increased and peaked in number by day 3, gradually returning to basal levels by day 28. FR-np-treated muscle, however, had significantly more satellite cells than the controls at day 3 and day 7, with an over-two-fold increase compared with vehicle alone. Further analysis showed that almost all Pax7⁺ cells were proliferating at day 3 (FIG. 10C). To directly determine whether these expanded Pax7⁺ cells were derived from existing Pax7⁺ satellite cells, the muscle injury and FR-np delivery experiments were performed in Pax7-CreER:Rosa26-EYFP mice, which allowed lineage tracing of Pax7⁺ cells with EYFP. FR-np treated muscle contained more EYFP⁺ cells at day 3 (FIG. 10D), providing evidence that FR-np increased the proliferation and expansion of existing Pax7⁺ cells in the injured muscle for enhanced muscle regeneration. In addition, the FR-np-treated muscles of both adult and aged mice exhibited significantly less muscle fibrosis and fewer macrophages than the vehicle-treated groups (Fang et al., Nat Biomed Eng. 2021 Mar. 18. doi: 10.1038/s41551-021-00696-y), demonstrating that administering the FR cocktail can modulate fibrosis and inflammation to improve muscle regeneration, similar to our findings with CiMC transplantation.

### Discussion

In this study, we have demonstrated a cocktail of chemicals that selectively and robustly expand myogenic stem cells from dermal cells and MuSCs for the regeneration of injured adult, aged and dystrophic muscles. In addition, in situ delivery of these chemicals to CTX-injured adult and aged muscles was achieved by using a nanoparticle system, which harnessed the body's innate regenerative potential to promote muscle regeneration. This approach of using small molecules is advantageous over genetic approaches in terms of its easy scalability, higher reproducibility as well as clinical safety. Both cell transplantation and drug delivery approaches have great potential to be translated into clinical applications.

Previously several approaches have been explored to address the unmet needs in skeletal muscle regeneration, including biomaterials^{4,5}, gene-editing^{28, 29} and stem cell-based strategies⁹. Satellite cells are essential in these approaches to achieve muscle regeneration, however, their expansion and self-renewal potential are limited in adult muscle, especially decreased or exhausted in aged^{7, 30, 31} and DMD muscle³². In addition, myogenic stem cells can be derived from somatic stem cells including bone marrow mesenchymal stem cells³³, umbilical cord blood mesenchymal stem cells³⁴ and mesoangioblasts³⁵, but the differentiation efficiency remains to be improved. Pluripotent stem cells including embryonic stem cells (ESCs) and induced pluripotent stem cells (iPSCs) may provide unlimited sources for myogenic cells³⁶⁻³⁸, and specifically, iPSCs can be used to generate myogenic cells without the ethical controversies of utilizing ESCs. However, this approach is still limited by the lengthy and expensive reprogramming and differentiation processes, and iPSC-derived myogenic cells are immature for efficient engraftment^{37, 38}. Our findings on CiMCs and FR cocktail help address these challenges.

Skin dermal cells have often been chosen as a cell source for cell reprogramming and therapies because they can be conveniently isolated via minimally invasive procedures. Here, we identified a chemical cocktail based on forskolin (F; a cAMP activator) and RepSox (R; a TGF-β1 inhibitor) that, in combination with bFGF and ascorbic acid, selectively induced and robustly expanded myogenic stem cells from dermal cells and MuSCs in vitro. Previous work demonstrated other relevant effects of F or R on myogenic proliferation and differentiation from ESCs and iPSCs^{37,39}. In addition, F and R as a part of a chemical cocktail or in combination with transcription factors can enhance or induce cell reprogramming, such as the conversion of human fibroblasts into neuronal⁴⁰, cardiac⁴¹, skeletal muscle⁴² and iPSCs^{20, 43}. Unlike other studies, however, we found that the combination of F and R robustly expanded CiMCs from dermal cells, while the efficiency is almost negligible when using F or R alone. On the other hand, dermal cell populations are highly heterogeneous and exhibit anatomic and developmental variation⁴⁴. Thus, different cell subpopulation(s) in dermal cells may contribute differently to chemical-induced myogenic induction and expansion. To clarify this issue, skin dermal cells were fractionated into SAC, RAC and HFC subpopulations, and we found that the enriched myogenic cells in SACs were highly correlated with chemical-mediated induction. We then performed lineage tracing and FACS sorting to show that CiMCs were primarily expanded from dermal Pax7⁺ cells by the FR medium. Furthermore, scRNA-seq analysis revealed the heterogeneity of dermal cells and the selective expansion of CiMCs. These findings provide a rational basis for using the chemical-expanded stem cells and drug delivery-based approaches for skeletal muscle regeneration.

Aged and DMD patients often suffer progressive muscle weakness and regenerative failure, due to the misregulation of satellite cells in the microenvironment⁴⁵. Recent studies have demonstrated the efficacy of muscle stem cell transplant in restoring muscle functions in aged and mdx mice^{9, 46, 47}. To our knowledge, this investigation is the first to use chemical-induced myogenic stem cells from dermal cells for injured aged and dystrophic muscle regeneration. Under optimized conditions, a large number of myogenic stem cells can be obtained from dermal cells through chemical induction and expansion. These in vitro expanded CiMCs can efficiently engraft into aged and mdx muscles, and significantly improve muscle functions and regeneration after 4 weeks of transplantation. Thus, CiMC transplantation may offer great potential for the treatment of patients suffering from age-related muscle dysfunction and inherited muscle diseases, in combination with gene editing technology. Before clinical application, further studies are needed, e.g., the scalability of ciMCs production and long-term evaluations of myotube survival and muscle functions.

Another highlight of this work is the development of drug-loaded nanoparticles for in situ satellite cell expansion and muscle regeneration. Notably, FR-np can be conveniently injected into injured TA muscles, whereby the controlled release of chemicals can effectively modulate local satellite cell numbers and functions to promote the regeneration of damaged muscles, especially for aged muscle regeneration. Previous investigations have shown that pathological muscle fibrosis can significantly retard muscle regeneration^{9, 43, 47}. We found that FR-np treatments had additional beneficial effects on muscle regeneration by reducing fibrosis, possibly due to the effects of TGF-β inhibition by RepSox⁴⁸⁻⁴⁹.

It is worth noting that, besides satellite cells and their progeny, immune system plays a crucial role in mediating muscle repair through spatial and temporal regulation of immune cells and cytokine secretion⁵⁰. For example, proinflammatory M1 macrophages appear soon after injury, which can remove apoptotic cells and necrotic fibers and stimulate satellite cell proliferation, whereas anti-inflammatory M2 macrophages play a role in regeneration phase and promote myoblast differentiation and muscle repair⁵¹. Indeed, the incorporation of macrophages into engineered tissues and the modulation of macrophage phenotype can enhance myogenesis and muscle regeneration^{52,53}. In aged and DMD mice, immune cells may cause a dysregulation of regeneration paradigm, and tuning macrophage phenotype improves muscle function⁵⁴. In our studies, CiMC- or FR cocktail-treated muscles showed faster and better regeneration with lower number of macrophages in adult, aged and DMD mice after 4 weeks. Furthermore, FR cocktail may have additional beneficial effects on immunomodulation, which is supported by the findings that elevated cAMP signaling and TGF-β inhibition can regulate macrophage and other innate and adaptive immune cells for muscle regeneration^{55, 56}. The immunomodulation effects of FR cocktail and the role of immune cells in the expansion and differentiation of myogenic cells during muscle regeneration require further mechanistic investigations. Overall, the approach that we have developed harnesses and maximizes the regenerative potential of resident cells to accelerate and promote muscle regeneration, which has great translational potential for clinical therapies.

### Materials and Methods

### Materials

Poly(D,L-lactide-co-glycolide) polymer (50:50, IV 0.4 dl/g) and Poly(vinyl alcohol) (PVA, MW 25000, 88% hydrolyzed) were purchased from Polysciences Inc. Poly(ethylene glycol) methyl ether-block-poly(lactide-co-glycolide) (PLGA-b-PEG, PEG average Mn 5,000, PLGA Mn 55,000), and dichloromethane were purchased from Sigma. Small molecules were purchased from Cayman Chemical.

### Mice

The mice strains used in this study were obtained from Jackson Laboratories, including C57BL/6J mice (Stock no. 000664, adult mice at 8 weeks and aged mice at 18 months), mdx mice (C57BL/10ScSn-Dmdmdx/J, Stock No: 001801), Rosa26-tdTomato (Stock no. 7909), Pax7-cre/ERT2 (Stock no. 017763), and Rosa26-EYFP (Stock no. 006148). Pax7-cre/ERT2 and Rosa26-EYFP were crossed to produce Pax7-CreER:Rosa26-EYFP offspring. The genotypes of all transgenic mice were confirmed with genotyping analyses according to the manufacturer's instructions. All mice were bred and maintained in specific pathogen-free conditions. All animal work was conducted under protocols approved by the UC Berkeley or UCLA Animal Research Committee.

### Cell Isolation and Culture

Primary murine neonatal dermal fibroblast-like cells from C57BL/6J and Pax7-CreER:Rosa26-EYFP mice were isolated as previously described⁵⁷. Briefly, the limbs and tail were removed from the sacrificed newborns (1-3 days) before gently pulling away the skin from the body. The skin was then flattened and floated on freshly thawed trypsin (0.25% without EDTA, Thermo Fisher Scientific) overnight and the dermis was separated from the epidermis the next day. The dermis was cut into small pieces and digested with 0.35% collagenase II in a 37° C. water bath for 1 hour. The digested mixture was filtered through a 100 μm mesh, centrifuged at 1000 rpm for 5 minutes and then washed twice with Dulbecco's Modified Eagle Medium (DMEM). The dermal cell pellet was plated and incubated at 37° C. in a humidified, 5% CO₂ incubator overnight in fibroblast culture medium (Fb medium, high-glucose DMEM containing 10% fetal bovine serum (FBS) and 1% penicillin/streptomycin). The following day the resulting mixed population of dermal cells was frozen into aliquots.

The mixed population of dermal cells was selected and separated into rapidly adhering cell (RAC) and slow adhering cell (SAC) subpopulations using a modified pre-plating technique⁵⁸. Briefly, dermal cells were plated onto a tissue culture-treated flask for 40 minutes. The attached cells represented the RAC fraction, whereas the non-adhering cells in the supernatant were transferred into another flask and cultured as the SAC fraction. Neonatal HFC⁵⁷, adult dermal fibroblasts⁵⁹ and adult and aged MuSCs⁶⁰ were isolated from C57BL/6J mice as previously described. The adult mice used for cell isolation were 4-8 weeks old and all these cells were cultured in Fb medium prior to being utilized for experiments.

### Screening Small-Molecules for Myogenic Induction

Small molecules to selectively expand skeletal muscle cells were screened using neonatal dermal fibroblast-like cells from C57BL/6J mice that were seeded at a density of 10,000 cells/cm² in 24-well plates containing Fb medium. The next day the original medium was replaced with a screening medium containing small-molecule cocktails: KnockOut DMEM (Thermo Fisher Scientific), 10% knockout serum replacement (Thermo Fisher Scientific), 10% FBS (Hyclone, Inc.), 2 mM GlutaMAX (Thermo Fisher Scientific), 1% nonessential amino acids (Thermo Fisher Scientific), 1% penicillin-streptomycin (Thermo Fisher Scientific) and 20 ng/ml bFGF (Stemgent) containing the various small molecules, including valproic acid (V, 500 μM), CHIR99021 (C, 20 μM), RepSox (R, 10 μM), tranylcypromine (T, 5 μM), forskolin (F, 10 μM), and 5-aza-2′deoxycytidine (5-Aza) (5-aza, 5 μM). The medium was changed once every 2-3 days. After optimizing the cocktail of small molecules and concentrations, the screening medium was replaced with Fb medium for further screening of additional candidates that may improve myogenic efficiency, including ascorbic acid (50 μg/ml, Sigma), BMP4 (20 ng/ml, Stemgent), Insulin (10 μg/ml, Stemgent), IGF-1 (50 ng/ml, R&D Systems), PDGF (50 ng/ml, R&D Systems), and bFGF (50 ng/ml, Stemgent Inc.).

Based on the results from the screening experiments, the optimized formulation was obtained and it consisted of Fb medium with 20 μM F, 20 μM R, 50 μg/ml AA, and 50 ng/ml bFGF, termed as “FR medium”. To determine which dermal cell subpopulations were involved in chemical-induced myogenesis, various subpopulations were tested for myogenesis potential in FR medium. For these experiments, cells were seeded at a density of 10 000 cells/cm² and cultured in Fb medium. The following day, the medium was replaced with FR medium. The medium was changed once every 2-3 days. To study the effect of passaging on the myogenic expansion potential of CiMCs, CiMCs were passaged every 3 days in FR medium. Meanwhile, a portion of the cells from all passages was stored by freezing. The passaged CiMCs were then treated with FR medium for 8 days, at which point immunofluorescence analysis of Pax7 and skeletal muscle markers was performed to evaluate the generation of myogenic cells. For dermal cells derived from Pax7-CreER:Rosa26-EYFP mice, 1 μM 4-OHT was added in Fb medium to induce Cre recombinase expression during cell seeding and then the medium was replaced with FR medium the following day.

### Flow Cytometry Analysis of Dermal Cells

Dermal cells in suspension were stained with antibodies such as FITC conjugated CD 90.2 (rat mAb, Thermo Fisher Scientific, 11-0903-81), P75 (rabbit pAb, Abcam, ab8874) and PDGFR-α (rat mAb, Thermo Fisher Scientific, 13-1401-82) (and appropriate secondary antibodies as needed), followed by flow cytometry analysis.

### Fluorescence-Activated Cell Sorting (FACS) of EYFP Reporter Cells

Neonatal dermal cells isolated from Pax7-CreER:Rosa26-EYFP mice were seeded in 10-cm Corning tissue-culture dishes at a cell density of 2×10⁴ cells/cm². 1 μM 4-OHT was added in the Fb medium during cell seeding to induce EYFP expression from Pax7 cells. One day later, cells were washed with PBS twice and FR medium was added and changed once on day 2. On day 4, cells were dissociated with Accutase and neutralized by FBS-containing media. Detached cells were centrifuged at 1000 rpm for 5 minutes and then resuspended in the sorting solution (DMEM containing 25 mM HEPES, 2% FBS and 1% penicillin/streptomycin) at a cell concentration of 5×10⁶ cells/mL after passing cells through a 40 μm filter to remove cell clusters and debris. Single-cell suspensions were kept on ice until sorting, and dermal cells without the presence of 4-OHT and FR were used as a negative control. EYFP⁺ and EYFP⁻ cells were sorted on a FACS Aria II instrument (Becton-Dickinson) after adding DAPI to exclude dead cells and collected in sorting solution. The sorted cells were re-plated in Fb medium and fixed at day 0 (i.e., 6 hours), or cultured in FR medium for 4 days, followed by immunofluorescence analysis of Pax7 and muscle marker expression.

### Microarray Analysis

Dermal fibroblast-like cells were treated with basal medium (Fb medium with 50 μg/ml AA and 50 ng/ml bFGF) and FR medium for 2 days. mRNA was extracted with RNeasy Micro Kit (Qiagen) and checked for RNA quality (RIN>7.5) with Bioanalyzer 2100 (Agilent) before linear amplification using Ovation Pico WTA System V2 (NuGEN). Biological triplicates were each hybridized to an Affymetrix Mouse Gene 1.0 ST Array and analyzed with GeneChip® Scanner 3000. CEL files were loaded into R and normalized with the RMA method using the oligo package. A linear model was fitted to each gene and empirical Bayes statistics calculated with the limma package. P-values for multiple testing were adjusted by the Benjamini-Hochberg method. Genes that were more than 2-fold different in expression level with adjusted P-values less than 0.05 were considered differentially expressed. Differentially expressed genes were submitted to DAVID for gene ontology enrichment analysis.

### Gene Expression Analysis

At the indicated time points, cells were lysed with Trizol (Thermo Fisher Scientific), and RNA was extracted following the manufacturer's instructions. The RNA concentration was quantified by absorption at 280 nm (Nanodrop 1000, Thermo Fisher Scientific), and an equal amount was loaded for cDNA synthesis using Maxima First Strand cDNA Synthesis Kit (Thermo Fisher Scientific). cDNA was then loaded into 96 well PCR plates with primers and Maxima SYBR Green qPCR Master Mix (Thermo Fisher Scientific). B2M was used as a housekeeping gene for normalization. Thermal cycling and data acquisition were performed on a CFX96 Real-Time PCR Detection System (Bio-Rad). Data were analyzed with the ΔΔCt method. Primers for RT-qPCR were used.

### Single-Cell Sequencing and Data Processing

Transcripts were mapped to the mm10 reference genome using Cell Ranger Version 3.1.0. Quality control was performed by selecting for cells with more than 1000 features and less than 50000 UMI counts. After quality control, 9433, 8034, 10326 and 6729 cells were retained for freshly isolated neonatal dermal cells (Neo DC), neonatal dermal cells treated with FR medium for 3 days (Neo DC/FR), adult dermal cells treated with FR medium for 3 days (Adult DC/FR) and freshly isolated endogenous adult MuSCs (Adult MuSC), respectively.

Cells from each sample were clustered using Seurat²⁴. In brief, data were log-normalized and highly variable genes were identified based on a variance stabilizing transformation. Data were scaled and centered before principal component analysis (PCA) was performed on the top 2000 most highly variable genes. The top 30 PCs were used for clustering using a shared nearest neighbor modularity optimization-based clustering algorithm with a resolution setting of 0.5. Differential gene expression testing was performed based on a hurdle model as implemented in the MAST package⁶¹. Data from different samples were integrated with Seurat by projecting the expression data into a lower dimension through canonical correlation analysis, identifying cells with similar biological states and then calculating and applying a transformation vector to all cells. Non-integrated data were used for differential expression testing between skeletal muscle cells from adult hindlimb and from dermal cells treated with FR. Genes with adjusted p values (Benjamini & Hochberg correction) less than 0.01 and log fold change more than 0.5 were considered differentially expressed.

For pseudotime analysis, skeletal muscle cells from the 4 samples were integrated and clustered with Seurat. A set of highly variable genes were identified by identifying the differentially expressed genes between these clusters. Dimensionality reduction using DDRTree was performed on these genes and a pseudotime trajectory was plotted using Monocle 2.12²⁵.

### Cell Transplantation

Twenty-four hours before cell transplantation, adult C57BL/6 mice (8 weeks), aged C57BL/6 mice (18 months) and mdx mice (8 weeks) were anesthetized with isoflurane/oxygen inhalation, and 30 μl of 20 μM Naja mossambica cardiotoxin (Sigma) in PBS was injected into the TA muscle of anesthetized mice to induce injury. CiMCs (1×10⁵ cells) were then suspended in 30 μl of Matrigel solution and injected directly into the pre-injured TA muscles. As a control, the contralateral muscles of recipient mice were similarly injured but injected with dermal cells cultured in Fb medium for 8 days. All transplanted cells were transduced with DsRed retrovirus for tracing before chemical induction with FR medium. Five animals per group were used for each time point.

### Preparation and Characterization of Drug-Loaded Nanoparticles (FR-Np)

Drug-loaded nanoparticles (FR-np) were prepared by an emulsification solvent evaporation technique⁶². Briefly, PLGA/PLGA-b-PEG (50/50 wt/wt) was dissolved in dichloromethane to make 10% w/v solutions, then 5% (wt/wt) of chemicals (F and R with the same molar ratio) to the polymer weight were co-dissolved in the polymer solution. The resulting solution was added to a stirred 1% (w/v) PVA solution using a vortex mixer at 2000 rpm for 2 minutes and then sonicated with a 20% amplitude (Sonic Dismembrator 500, Thermo Fisher Scientific) for 40 seconds. After sonication, the emulsion was added dropwise into 1% PVA and stirred for 3 hours at room temperature to remove the residual organic solvent. The nanoparticles were collected and washed three times with distilled water by centrifugation at 10,000×g for 5 minutes at 4° C. Particle diameter was measured by dynamic light scattering (DLS) and the surface morphology was observed by SEM with gold electrospray.

### Characterization of Drug Release Profile

Briefly, 2 mg of FR-nps was dispersed in a 0.22 μm filter inserted into a centrifuge tube (Corning™ Costar™ Spin-XT™ Centrifuge Tube, Thermo Fisher Scientific) with 1 ml PBS (pH 7.4) at 37° C., with continuous shaking. At discrete time intervals (16 hours, 1, 2, 4, 6, 8, 12, and 16 days), 0.5 ml of the sample solution was collected from the tube and frozen for later analysis. Aliquots of the solutions were analyzed by reversed-phase separation and detection using tandem mass spectrometry with multiple reactions monitoring with previously optimized conditions for parent ion production and fragment ion detection on a triple quadrupole mass spectrometer (Agilent 6460). Quantification was achieved with the external standards of both analytes. All experimental samples were analyzed in triplicate and all results were reported as mean f standard error of the mean.

In Vitro Myogenesis with Drug-Loaded Particles

For selective induction of myogenic cells in dermal cells or MuSCs using FR-nps, the dermal cells were seeded in 24-well plates at 10 000 cells/cm² with Fb medium. The next day, the medium was replaced with Fb medium containing 50 μg/ml AA and 50 ng/ml bFGF. Meanwhile, FR-nps at various doses (1 mg, 2 mg, and 4 mg) were added into the inserted Transwells (0.4 μm pore size, Thermo Fisher Scientific) of the co-culture system. Half of the Fb medium was changed every other day.

In Situ Regeneration with Drug-Loaded Particles

For in situ regeneration, adult C57BL/6 mice (8 weeks) and aged C57BL/6 mice (18 months) were used and injured with CTX injection as described above, and 1 mg drug-loaded particles (FR-np) suspended in 30 μl PBS was injected into the injured TA muscle. As a control, the contralateral muscles of recipient mice were similarly injured but injected with nps without drugs. Six animals per group were used for each time point. Pax7-CreER:Rosa26-EYFP transgenic mice were used for lineage tracing of Pax7⁺ satellite cells. Before performing the same procedures, 100 μl of 10 mg/ml tamoxifen (Sigma, T5648) diluted in corn oil (Sigma, C8267) was intraperitoneally injected for 5 consecutive days. Seven days after the last injection, anesthesia, CTX injury and FR-np injection were performed. Three animals per group were used for each time point.

To visualize the distribution of nanoparticles in injured muscles after injection, green fluorescence-labeled nanoparticles (green-nps) were prepared according to the same protocol for making FR-nps, only with a minor modification, which involved adding 0.02% (wt) coumarin-6 (green fluorescence, Sigma) in the polymer solution for green-nps fabrication. Similar to FR-np injection for muscle therapy, 1 mg green-nps were injected into injured TA muscles of adult C57BL/6 mice (8 weeks). Muscle samples were collected after 2 days and 1 month, and cross and longitudinal cryosectioned before performing immunofluorescence analysis and imaging.

### Electrophysiological Analysis

Before harvesting muscle samples, CMAPs of each TA muscle were measured after stimulating the sciatic nerve in hindlimbs using needle electrodes as previously described⁶³. In brief, the murine sciatic nerve was exposed to electrical stimuli (single-pulse shocks, 1 mA, 0.1 ms) and CMAPs were recorded on the gastrocnemius belly from 1 V. Normal CMAPs from the contralateral side of the sciatic nerve were also recorded for comparison. Grass Tech S88X Stimulator (Astro-Med, Inc.) was used for the test and PolyVIEW16 data acquisition software (Astro-Med, Inc.) was used for the recording.

### Force Measurement

The isometric tetanic force of all mice TA muscles were measured with a commercial device (Grass Tech, Astro-Med Inc) as previously described⁶⁴. Briefly, mice were anesthetized by isoflurane and warmed by a heating lamp during the entire procedure, the tendon was exposed and attached to a force transducer (Grass FT03 Transducer, Astro-Med Inc), and the knee was immobilized by a stainless-steel pin. The electrical stimulation was performed via a bipolar electrode with a Grass stimulator to the sciatic nerve. The maximum isometric tetanic force was achieved by applying single-pulse stimuli (volts=12 V, duration=0.2 ms, pulse rate=100 Hz) at an optimal muscle length, which was adjusted with 0.5 mm increments. Data were acquired and recorded with the PolyVIEW16 software (Grass Tech, Astro-Med Inc.). Following the completion of the isometric force testing, the mouse was euthanized, and the entire TA muscle was carefully dissected and weighed.

### Muscle Sample Collection

TA muscles were harvested at various time points, and fresh frozen by liquid nitrogen-cooled isopentane (Sigma) for 1 minute. Muscles from mice implanted with DsRed-labeled cells and Pax7-CreER:Rosa26-EYFP mice were fixed at room temperature for 2 hours in 1% paraformaldehyde, and dehydrated with 20% sucrose overnight at 4° C., followed by OCT embedding and freezing in liquid nitrogen-cooled isopentane. The samples were cryo-sectioned to obtain 12 μm thick cross-sections and collected on pre-warmed, positively charged microscope slides.

### Histological Analysis

H&E staining was performed on muscle cryosections to determine tissue histology using bright field microscopy. The cross-sectional area (CSA) of myofibers in mid-belly sections of muscle samples was measured by ImageJ based on H&E staining slides. Masson's Trichrome staining was performed using standard protocols, and the total fibrotic area within a section was quantified with a threshold intensity program from ImageJ. The fibrotic index was calculated as the area of fibrosis divided by the total area of muscle.

### Immunofluorescence Staining

For cell immunostaining, cells were fixed in 4% paraformaldehyde for 15 minutes and permeabilized with 0.5% Triton-X 100 in PBS for 15 minutes. Cells were then blocked with 5% donkey serum for 1 hour and incubated overnight at 4° C. with primary antibodies (diluted in 5% donkey serum), including TnT (mouse mAb, DSHB), Myh1E (MF 20, mouse mAb, DSHB), Myh2 (SC-71, mouse mAb, DSHB), Myh3 (F1.652, mouse mAb, DSHB), Myh4 (BF-F3, mouse mAb, DSHB), Myh7 (BA-D5, mouse mAb, DSHB), Myh8 (Rabbit pAb, Thermo Fisher Scientific, PA5-72846), MANEX1011B(1C7) (dystrophin, mouse mAb, DSHB), MyoD (mouse mAb, DSHB), MyoG (mouse mAb, DSHB), Pax7 (mouse mAb, DSHB), Sox 10 (goat pAb, R&D Systems, AF2864), Ki 67 (rabbit mAb, Abcam, ab16667), FSP1 (rabbit mAb, Sigma, 07-2274), CD 90.2-FITC (rat mAb, Thermo Fisher Scientific, 11-0903-81), P75 (rabbit pAb, Abcam, ab8874) and PDGFR-α (rat mAb, Thermo Fisher Scientific, 13-1401-82). Then appropriate Alexa Fluor 488- or Alexa Fluor 546- or Alexa Fluor 647-conjugated secondary antibodies (Thermo Fisher Scientific) were used for 1 hour at room temperature. Thereafter, nuclei were stained with 4′,6-diamindino-2-phenylindole (DAPI, Sigma) for 10 minutes in the dark.

For immunohistological staining, the same protocol was used with minor modifications. Mid-belly transverse sections (10 μm thickness) were fixed in 4% (vol/vol) paraformaldehyde for 10 minutes and washed with PBS for 5 minutes (3 times), then permeabilized with 0.5% (vol/vol) Triton X-100 (Sigma) for 10 minutes. Slices were then blocked with 5% donkey serum in 0.1% (vol/vol) Triton X-100 for 1 hour and incubated overnight at 4° C. with primary antibodies (diluted in 5% donkey serum), including anti-laminin (rabbit mAb, Sigma, L9393), anti-Pax7 (mouse mAb, DSHB), anti-Ki67 (rabbit mAb, Abcam, ab16667), and F4/80 (rat mAb, Abcam, ab6640). For Pax7 staining, heat-activated antigen retrieval was performed by placing the paraformaldehyde-fixed samples in citrate buffer (pH 6.0) at 95° C. for 20 minutes and cooling the slides at room temperature for 20 minutes, followed by permeabilization and blocking before co-staining with other antibodies as mentioned above. For myofiber staining, fresh frozen sections were used for staining with primary antibodies, including BA-D5 concentrate (myosin heavy chain type I, mouse mAb. DSHB), SC-71 concentrate (myosin heavy chain type HA, mouse mAb, DSHB), BF-F3 concentrate (myosin heavy chain type IB, mouse mAb, DSHB), and laminin (rabbit mAb, Sigma, L9393) and then stained with secondary antibodies, including DyLight™ 405 AffiniPure goat anti-mouse IgG2b, Alexa Fluor® 488 AffiniPure goat anti-mouse IgG1, Alexa Fluor® 594 AffiniPure goat anti-mouse IgM (all from Jackson ImmunoResearch Laboratories, catalog numbers 115-475-207, 115-545-205, and 115-585-075, respectively), and Alexa Fluor® 647 donkey anti-rat IgG (Thermo Fisher Scientific, ab150155). All fluorescent images were taken with a Zeiss Axio Observer Z1 inverted microscope and a confocal inverted Leica TCS-SP8-SMD confocal microscope.

### Statistical Analysis

Values are expressed as means t SD calculated from the average of at least three biological replicates unless otherwise specified. The statistical significance of differences was estimated by one-way ANOVA with a t-test, using Origin 8 software. P-value<0.05 was considered significant.

### REFERENCES

1. Pedersen. B. K. & Febbraio, M. A. Muscles, exercise and obesity: skeletal muscle as a secretory organ. Nat. Rev. Endocrinol. 8, 457-465 (2012).
2. Seale, P. et al. Pax7 is required for the specification of myogenic satellite cells. Cell 102, 777-786 (2000).
3. Yin, H., Price, F. & Rudnicki, M A. Satellite cells and the muscle stem cell niche. Physiol. Rev. 93, 23-67 (2013).
4. Sicari, B. M. et al. An acellular biologic scaffold promotes skeletal muscle formation in mice and humans with volumetric muscle loss. Sci. Transl. Med 6 (2014).
5. Quarta, M. et al. Bioengineered constructs combined with exercise enhance stem cell-mediated treatment of volumetric muscle loss. Nat. Commun. 8 (2017).
6. Schworer, S. et al. Epigenetic stress responses induce muscle stem-cell ageing by Hoxa9 developmental signals. Nature 540, 428-432 (2016).
7. Chakkalakal. J. V., Jones. K. M., Basson, M. A. & Brack, A. S. The aged niche disrupts muscle stem cell quiescence. Nature 490.355-360 (2012).
8. Mercuri, E. & Muntoni, F. Muscular dystrophies. The Lancet 381, 845-86((2013).
9. Cerletti, M. et al. Highly efficient, functional engraftment of skeletal muscle stem cells in dystrophic muscles. Cell 134, 37-47 (2008).
10. Montarras, D. et al. Direct isolation of satellite cells for skeletal muscle regeneration. Science 309, 2064-2067(2005).
11. Sacco, A., Doyonnas. R., Kraft, P., Vitorovic, S. & Blau, H. M. Self-renewal and expansion of single transplanted muscle stem cells. Nature 456, 502-506 (2008).
12. Davis, R. L., Weintraub, H. & Lassar, A. B. Expression of a single transfected cDNA converts fibroblasts to myoblasts. Cell 51, 987-1000 (1987).
13. Qiu, Z. et al. Skeletal myogenic potential of mouse skin-derived precursors. Stem Cells Dev. 19, 259-268 (2010).
14. Galli, R. et al. Skeletal myogenic potential of human and mouse neural stem cells. Nat. Neurosci. 3, 986-991 (2000).
15. Li, W. L., Li, K., Wei, W. G. & Ding, S. Chemical approaches to stem cell biology and therapeutics. Cell stem cell 13, 270-283 (2013).
16. Zismanov, V. et al. Phosphorylation of eIF2 alpha is a translational control mechanism regulating muscle stem cell quiescence and self-renewal. Cell stem cell 18, 79-90 (2016).
17. Xu, C. et al. A zebrafish embryo culture system defines factors that promote vertebrate myogenesis across species. Cell 155, 909-921 (2013).
18. Bernet, J. D. et al. p38 MAPK signaling underlies a cell-autonomous loss of stem cell self-renewal in skeletal muscle of aged mice. Nat. Med 20, 265-271 (2014).
19. Fu, X. et al. Combination of inflammation-related cytokines promotes long-term muscle stem cell expansion. Cell Res. 25. 655-673 (2015).
20. Hou, P. P. et al. Pluripotent stem cells induced from mouse somatic cells by small-molecule compounds. Science 341, 651-654 (2013).
21. Constantinides. P. G., Jones. P. A. & Gevers. W. Functional striated-muscle cells from non-myoblast precursors following 5-azacytidine treatment. Nature 267, 364-366 (1977).
22. Chen, J. et al. Vitamin C modulates TETI function during somatic cell reprogramming. Nat. Genet. 45, 1504-1509 (2013).
23. Esteban, M. A. et al. Vitamin C enhances the generation of mouse and human induced pluripotent stem cells. Cell stem cell 6, 71-79 (2010).
24. Butler, A., Hoffman, P., Smibert, P., Papalexi. E. & Satija, R. Integrating single-cell transcriptomic data across different conditions, technologies, and species. Nat. Biotechnol. 36, 411-420 (2018).
25. Trapnell, C. et al. The dynamics and regulators of cell fate decisions are revealed by pseudotemporal ordering of single cells. Nat. Biotechnol. 32, 381-386 (2014).
26. Schiaffino, S. & Reggiani. C. Fiber types in mammalian skeletal muscles. Physiol. Rev. 91, 1447-1531 (2011).
27. Acuna, L. et al. Morphometric and histopathologic changes in skeletal muscle induced for injectable plga microparticles. Int. J. Morphol. 29, 403-408 (2011).
28. Long, C. et al. Postnatal genome editing partially restores dystrophin expression in a mouse model of muscular dystrophy. Science 351, 400-403 (2016).
29. Amoasii, L. et al. Gene editing restores dystrophin expression in a canine model of Duchenne muscular dystrophy. Science 362, 86-91 (2018).
30. Blau, H. M., Cosgrove. B. D. & Ho, A. T. V. The central role of muscle stem cells in regenerative failure with aging. Nat. Med 21, 854-862 (2015).
31. Sousa-Victor, P. et al. Geriatric muscle stem cells switch reversible quiescence into senescence. Nature 506. 316-321 (2014).
32. Boonen, K. J. & Post. M. J. The muscle stem cell niche: regulation of satellite cells during regeneration. Tissue Eng. Part B Rev. 14, 419-431 (2008).
33. Ferrari, G. et al. Muscle regeneration by bone marrow-derived myogenic progenitors. Science 279, 1528-1530 (1998).
34. Gang. E. J. et al. Skeletal myogenic differentiation of mesenchymal stem cells isolated from human umbilical cord blood. Stem cells 22, 617-624 (2(004).
35. Sampaolesi. M. et al. Cell therapy of alpha-sarcoglycan null dystrophic mice through intra-arterial delivery of mesoangioblasts. Science 301, 487-492 (2003).
36. Chal. J. et al. Differentiation of pluripotent stem cells to muscle fiber to model Duchenne muscular dystrophy. Nature biotechnology 33, 962-U207 (2015).
37. Hicks, M. R. et al. ERBB3 and NGFR mark a distinct skeletal muscle progenitor cell in human development and hPSCs. Nat. Cell Biol. 20, 46-57 (2018).
38. Young, C. S. et al. A single CRISPR-Cas9 deletion strategy that targets the majority of DMD patients restores dystrophin function in hiPSC-derived muscle cells. Cell stem cell 18. 533-540 (2016).
39. Oh, J., Lee. Y. D. & Wagers. A. J. Stem cell aging: mechanisms, regulators and therapeutic opportunities. Nat. Med 20, 870-880 (2014).
40. Hu, W. X. et al. Direct conversion of normal and Alzheimer's disease human fibroblasts into neuronal cells by small molecules. Cell stem cell 17, 204-212 (2015).
41. Fu, Y. B. et al. Direct reprogramming of mouse fibroblasts into cardiomyocytes with chemical cocktails. Cell Res. 25, 1013-1024 (2015).
42. Bar-Nur, O. et al. Direct reprogramming of mouse fibroblasts into functional skeletal muscle progenitors. Stem Cell Rep. 10, 1505-1521 (2018).
43. Zhao, Y. et al. A XEN-like state bridges somatic cells to pluripotency during chemical reprogramming. Cell 163, 1678-1691 (2015).
44. Singhal, P. K. et al. Mouse embryonic fibroblasts exhibit extensive developmental and phenotypic diversity. Proc. Natl Acad Sci. USA 113, 122-127 (2016).
45. Almada, A. E. & Wagers. A. J. Molecular circuitry of stem cell fate in skeletal muscle regeneration, ageing and disease. Nat. Rev. Mol. Cell Biol. 17, 267-279 (2016).
46. Cosgrove, B. D. et al. Rejuvenation of the muscle stem cell population restores strength to injured aged muscles. Nat. Med 20, 255-264 (2014).
47. Blau, H. M., Cosgrove, B. D. & Ho, A. T. The central role of muscle stem cells in regenerative failure with aging. Nat. Med 21, 854-862 (2015).
48. Ichida, J. K. et al. A small-molecule inhibitor of Tgf-beta signaling replaces Sox2 in reprogramming by inducing Nanog. Cell stem cell 5, 491-503 (2009).
49. Serrano, A. L. & Munoz-Canoves, P. Regulation and dysregulation of fibrosis in skeletal muscle. Experimental cell research 316, 3050-3058 (2010).
50. Tidball, J. G. Regulation of muscle growth and regeneration by the immune system. Nat. Rev. Immunol. 17, 165-178 (2017).
51. Tidball, J. G. & Wehling-Henricks, M. Shifts in macrophage cytokine production drive muscle fibrosis. Nat. Med 21. 665-666 (2015).
52. Juhas, M. et al. Incorporation of macrophages into engineered skeletal muscle enables enhanced muscle regeneration. Nat. Biomed Eng. 2, 942-954 (2018).
53. Raimondo, T. M. & Mooney, D. J. Functional muscle recovery with nanoparticle-directed M2 macrophage polarization in mice. Proc. Natl Acad. Sci. USA 115, 10648-10653 (2018).
54. Welc. S. S. et al. Targeting a therapeutic LIF transgene to muscle via the immune system ameliorates muscular dystrophy. Nat. Commun. 10. 2788 (2019).
55. Akhurst, R. J. & Hata, A. Targeting the TGFbeta signalling pathway in disease. Nat. Rev. Drug Discov. 11, 790-811 (2012).
56. Raker. V. K., Becker, C. & Steinbrink, K. The cAMP Pathway as therapeutic target in autoimmune and inflammatory diseases. Front. Immunol. 7, 123 (2016).
57. Lichti, U., Anders, J. & Yuspa, S. H. Isolation and short-term culture of primary keratinocytes, hair follicle populations and dermal cells from newborn mice and keratinocytes from adult mice for in vitro analysis and for grafting to immunodeficient mice. Nat. Protoc. 3, 799-810 (2008).
58. Gharaibeh, B. et al. Isolation of a slowly adhering cell fraction containing stem cells from murine skeletal muscle by the preplate technique. Nat. Protoc. 3, 1501-1509 (2008).
59. Seluanov, A., Vaidya. A. & Gorbunova, V. Establishing primary adult fibroblast cultures from rodents. J. Vis. Exp. (2010).
60. Liu, L., Cheung, T. H., Charville, G. W. & Rando, T. A. Isolation of skeletal muscle stem cells by fluorescence-activated cell sorting. Nature protocols 10, 1612-1624 (2015).
61. Finak. G. et al. MAST: a flexible statistical framework for assessing transcriptional changes and characterizing heterogeneity in single-cell RNA sequencing data. Genome biology 16. 278 (2015).
62. Danhier, F. et al. PLGA-based nanoparticles: An overview of biomedical applications. J. Control. Release 161, 505-522 (2012).
63. Huang, C. W. et al. The differentiation stage of transplanted stem cells modulates nerve regeneration. Sci. Rep. 7 (2017).
64. Bonetto, A., Andersson, D. C. & Waning. D. L. Assessment of muscle mass and strength in mice. Bonekey Rep. 4, 732 (2015).

Administration of the FR cocktail in an appropriate pharmaceutical composition, can be carried out via any of the accepted modes of administration of agents for serving similar utilities. The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in Remington's Pharmaceutical Sciences (Mack Pub. Co., N.J. current edition).

Persons skilled in the relevant arts will be familiar with any number of diagnostic, surgical and other clinical criteria to which can be adapted administration of the pharmaceutical compositions described herein. See, e.g., Humar et al., Atlas of Organ Transplantation, 2006, Springer; Kuo et al., Comprehensive Atlas of Transplantation, 2004 Lippincott, Williams & Wilkins; Gruessner et al., Living Donor Organ Transplantation, 2007 McGraw-Hill Professional; Antin et al., Manual of Stem Cell and Bone Marrow Transplantation, 2009 Cambridge University Press; Wingard et al. (Ed.), Hematopoietic Stem Cell Transplantation: A Handbook for Clinicians, 2009 American Association of Blood Banks; Sabiston, Textbook of Surgery, 2012 Saunders & Co.; Mulholland, Greenfield's Surgery, 2010 Lippincott, Williams & Wilkins; Schwartz's Principles of Surgery, 2009 McGraw-Hill; Lawrence, Essentials of General Surgery 2012 Lippincott, Williams & Wilkins.

All publications mentioned herein (e.g., Fang et al., Nat Biomed Eng. 2021 Mar. 18. doi: 10.1038/s41551-021-00696-y) are incorporated herein by reference to disclose and describe aspects, methods and/or materials in connection with the cited publications.

1. A composition of matter comprising a mammalian cell culture media, wherein the cell culture media comprises a supplement disposed therein consisting essentially of forskolin and RepSox.

2. The composition of claim 1, wherein the cell culture media supplement comprises amounts of forskolin and amounts of RepSox sufficient to induce placental cells, dermal cells, skeletal muscle stem cells or myogenic cells growing the cell culture media to expand a population of myogenic stem cells.

3. The composition of claim 2, wherein amounts of forskolin and amounts of RepSox are sufficient to create a concentration of forskolin that is between 1 μM and 100 μM and a concentration of RepSox that is between 1 μM and 100 μM in the environment in which the composition is disposed.

4. The composition of claim 1, further comprising:
ascorbic acid;
basic fibroblast growth factor; and/or
a pharmaceutically acceptable carrier.

5. The composition of claim 1, further comprising:
placental cells;
dermal cells;
skeletal muscle stem cells; and/or
myogenic stem cells.

6. A method of growing myogenic stem cells from placental cells, dermal cells and/or skeletal muscle stem cells comprising combining placental cells, dermal cells and/or skeletal muscle stem cells with amounts of forskolin and amounts of RepSox sufficient to induce and expand a population of myogenic stem cells from the placental cells, dermal cells, skeletal muscle stem cells.

7. The method of claim 6, wherein amounts of forskolin and amounts of RepSox are sufficient to generate at least 10% more Pax7⁺ myogenic stem cells and/or at least 10% more MyoD⁺ myogenic stem cells growing in an in vitro culture for at least 4 days as compared to a control comprising an at least 4-day in vitro culture of placental cells, dermal cells, skeletal muscle stem cells that lacks forskolin and RepSox.

8. The method of claim 6, wherein placental cells, dermal cells, skeletal muscle stem cells are further combined with amounts of ascorbic acid and/or basic fibroblast growth factor sufficient to enhance induction and expansion of the myogenic stem cells.

9. The method of claim 6, further comprising disposing the expanded myogenic stem cells at a site of injury in vivo.

10. The method of claim 9, wherein the site comprises skeletal muscle tissue.

11. A method of making a mammalian cell culture media, the method comprising combining together water, serum or growth factors, a buffering agent, an antibiotic agent and a supplement consisting essentially of forskolin and RepSox such that the mammalian cell culture media is made.

12. The method of claim 11, wherein the cell culture media supplement comprises amounts of forskolin and amounts of RepSox sufficient to induce placental cells, dermal cells or skeletal muscle stem cells growing the cell culture media to form myogenic stem cells.

13. The method of claim 12, wherein amounts of forskolin and amounts of RepSox are sufficient to create a concentration of forskolin that is between 1 μM and 100 μM and a concentration of RepSox that is between 1 μM and 100 μM in the environment in which the composition is disposed.

14. The method of claim 11, further comprising adding to the mammalian cell culture media:
ascorbic acid;
basic fibroblast growth factor; and/or
a pharmaceutically acceptable carrier.

15. A composition of matter comprising nanoparticles loaded with amounts of forskolin and amounts of RepSox sufficient to induce dermal cells and/or skeletal muscle stem cells to become myogenic stem cells when disposed in an in vivo environment comprising the dermal cells and/or the skeletal muscle stem cells.

16. The composition of claim 15, further comprising a pharmaceutically acceptable carrier.

17. The composition of claim 15, further comprising amounts of ascorbic acid and/or basic fibroblast growth factor sufficient to enhance induction and expansion of the myogenic stem cells.

18. The composition of claim 15, wherein the nanoparticles comprise biodegradable poly (D, L-lactide-co-glycolide).

19.-20. (canceled)
